(19) United States
(12) Patent Application Publication
(10) Pub. No.: US 2024/0241107 A1
(43) Pub. Date: Jul. 18, 2024

(54) SERUM METABOLOMICS RELATED TO CHIMERIC ANTIGEN RECEPTOR (CAR) T-CELL THERAPY

(71) Applicant: BOARD OF REGENTS, THE UNIVERSITY OF TEXAS SYSTEM, Austin, TX (US)

(72) Inventors: Neeraj SAINI, Sugarland, TX (US); Chia-Chi CHANG, Houston, TX (US); Johannes FAHRMANN, Houston, TX (US); Robert R. JENQ, Houston, TX (US); Samir HANASH, Houston, TX (US); Sattva S. LAPU, Houston, TX (US)

(73) Assignee: BOARD OF REGENTS, THE UNIVERSITY OF TEXAS SYSTEM, Austin, TX (US)

(21) Appl. No.: 18/559,329

(22) PCT Filed: May 6, 2022
(86) PCT No.: PCT/US2022/028184

§ 371 Date: Nov. 6, 2023

Related U.S. Application Data
(60) Provisional application No. 63/185,412, filed on May 7, 2021.
(60) Provisional application No. 63/257,621, filed on Oct. 20, 2021.

(51) Int. Cl. G01N 33/50; A61K 31/132; A61K 31/16; A61K 31/661; A61K 35/17; A61K 39/00; C12N 5/0783

(52) U.S. Cl. CPC G01N 33/505; A61K 31/132; A61K 31/16; A61K 31/661; A61K 35/17; A61K 39/4631; C12N 5/0636; C12N 2510/00; G01N 2800/52

(57) ABSTRACT

Disclosed herein are methods and compositions for determining and improving the likelihood of response and/or determining and decreasing the likelihood of toxicity to a cellular therapy using metabolomics. Embodiments of the disclosure include methods for measuring certain metabolites in biological samples of an individual for determining whether the individual has a responder or non-responder phenotype to the cellular therapy or to a toxicity of the cellular therapy. Embodiments also include compositions for increasing the response to a cellular therapy or decreasing toxicities associated with a cellular therapy. Embodiments also show that plasma metabolomics correlate with efficacy and toxicities associated with CAR-T therapy towards a cancer. The present disclosure relates to compositions and methods for predicting a subject's response to a CAR T-cell therapy, by analyzing the plasma metabolomics of the subject. The present disclosure further provides therapeutic compositions and methods for treating a subject having a cancer, to improve the efficacy of CAR T-cell therapy.

### CROSS-REFERENCE TO RELATED APPLICATIONS

This application claims priority to U.S. Provisional Patent Application Ser. No. 63/185,412, filed May 7, 2021, and also claims priority to U.S. Provisional Patent Application Ser. No. 63/257,621, filed Oct. 20, 2021, both of which applications are incorporated by reference herein in their entireties.

### STATEMENT REGARDING FEDERALLY SPONSORED RESEARCH OR DEVELOPMENT

This invention was made with government support under P30 CA016672 awarded by NIH-NCI. The government has certain rights in the invention.

### TECHNICAL FIELD

Embodiments of the disclosure encompass at least the fields of cell biology, molecular biology, immunology, and medicine.

### BACKGROUND

Considerable progress has been made in cancer therapeutics recently with targeted strategies that are efficacious and less toxic. Immunotherapy and chimeric antigen receptor (CAR) T-cells are increasingly being evaluated in a variety of tumors in the relapsed/refractory as well as frontline disease settings, predominantly in hematologic malignancies. Despite impressive outcomes in select patients, there remains significant heterogeneity in clinical response to CAR T-cells. Metabolites have emerged as one of the key host factors that could potentially be modulated to not only enhance responses to immunotherapy but also alleviate toxicities related to it. Several recent human studies evaluating immunotherapy strategies such as immune checkpoint inhibitor therapy showed a significantly superior response and survival in patients with the more diverse gut microbiome. Currently, it is unknown if plasma metabolomics modulates anti-tumor responses and toxicities related to CAR T-cells. Studies correlating the role of blood metabolites to outcomes of CAR-T therapy are lacking.

### BRIEF SUMMARY

The present disclosure is directed to systems, compositions, and methods encompassing metabolomics related to immunotherapies, such as adoptive cell therapies that include immune cells that express chimeric antigen receptors (CAR) or T-cell receptors. Certain embodiments encompass methods for treating an individual and methods for reducing risks for an individual receiving an immunotherapy, such as CAR-related therapies. Certain embodiments encompass decreasing the likelihood of toxicity of a cellular therapy. In some embodiments, the method comprises measuring one or more compositions in the individual, including before, during, and/or after receiving the therapy. The compositions may include biomarkers, metabolites, or the like, and in some cases one or more metabolites act as biomarkers related to efficacy and/or toxicity of a therapy for an individual. In specific embodiments, one or more metabolites acts as predictive biomarkers for outcomes associated with CAR-related therapies. In some embodiments, the metabolites comprise microbial-associated metabolites including indole and/or associated indole derivatives, and trimethylamine oxide (TMAO), or non-microbial metabolites including 1-methylnicotinamide, nicotinamide, a polyamine, asymmetric dimethylarginine, symmetric dimethylarginine, taurine, 6-phosphogluconic acid, triacylglycerol, N-alpha-L-acetyl L-asparagine, deoxycarnitine, or a combination thereof. The indole derivative may comprise tryptophan, serotonin, indole-3-acetaldehyde, indoleacrylic acid, indoxyl sulfate, or a combination thereof. The polyamine may comprise diacetylspermine, N8-acetylspermidine, putrescine, spermidine, spermine, acetylputresceine, acetylspermidine, acetylspermine, N3AP, cadaverine, acetylcadaverine, diacetylcadaverine or a combination thereof.

Certain embodiments of the disclosure concern methods for measuring compositions, such as one or more metabolites, in any biological sample from an individual. In some embodiments, the sample comprises a blood sample. The blood sample may comprise any component of blood, including whole blood, serum, and/or plasma. In particular embodiments, measuring of one or more metabolites results in measurements that are indicative whether an individual will or will not have toxicity in response to CAR-related therapy. In particular embodiments, measuring of one or more metabolites results in measurements that are indicative whether an individual will or will not have an efficacious therapeutic response to CAR-related therapy. In some embodiments, measuring the compositions identifies the individual as having a responder phenotype or as having a non-responder phenotype. Also included are embodiments wherein measurements indicate that a therapy should be modulated for an individual, including modulations such as reducing level of one or more metabolites in an individual or increasing level of one or more metabolites in an individual. In some cases for the same individual, the measurements determine that one or more metabolites need to have an increased level for the individual and one or more other metabolites need to have a decreased level for the individual.

Certain embodiments concern methods comprising administering an amount of a cellular therapy to an individual, including any individual encompassed herein. The amount may comprise a therapeutically effective amount of the cellular therapy. In some embodiments, the therapeutically effective amount is determined based on measuring levels of one or more metabolites from a sample from the individual. In some embodiments, the therapeutically effective amount is dependent on the individual having a responder phenotype or a non-responder phenotype. In certain embodiments, a responder phenotype comprises a concentration of at least one metabolite in the biological sample that is statistically equal to a concentration of the metabolite in biological samples, including any type of blood sample, of individuals known to respond to an amount of the cellular therapy. In certain embodiments, a non-responder phenotype comprises a concentration of at least one metabolite in the biological sample that is statistically higher than a concentration of the metabolite in biological samples, including any type of blood sample, of individuals known to respond to an amount of the cellular therapy. In certain embodiments, a non-responder phenotype comprises a concentration of at least one metabolite in the biological sample that is statistically lower than a concentration of the metabolite in biological samples, including any type of blood sample, of individuals known to respond to an amount of the cellular therapy. In some embodiments, such as when the individual has a non-responder phenotype, the individual does not receive the therapy, or the therapy is modulated and/or supplemented prior to delivery; such supplementation may include delivery of one or compositions capable of increasing or decreasing at least one of the metabolites in the individual.

In some embodiments, a blood sample is taken from an individual prior to, during, and/or after a cellular therapy is administered to the individual. At least one metabolite may be measured in the blood sample. In some embodiments, at least one metabolite is measured in a second blood sample. The second blood sample may comprise a blood sample taken from an individual that has been administered a therapy, including a cellular therapy. The individual may have a responder phenotype when the concentration of the metabolite(s) in the blood sample are equal to the concentration of the metabolite(s) in the second blood sample. The individual may have a non-responder phenotype when the concentration of the metabolite(s) in the blood sample are different the concentration of the metabolite(s) in the second blood sample.

Certain embodiments concern methods for decreasing the likelihood of toxicity of a cellular therapy in an individual. The cellular therapy may be any cellular therapy encompassed herein, including any CAR T-cell therapy. The toxicity may comprise cytokine response syndrome, immune effector cell-associated neurotoxicity, or a combination thereof. In some embodiments, the individual is monitored for the toxicity, including during and/or in some cases just after the therapy is delivered.

Certain embodiments encompass one or more cellular therapies. In some embodiments, the one or more cellular therapies comprise a T-cell therapy. In some embodiments, the one or more cellular therapies comprise a cell comprising at least one engineered receptor. The engineered receptor may comprise a chimeric antigen receptor (CAR). In some embodiments, the one or more cellular therapies comprise a cell comprising an enzyme, and/or a nucleic acid encoding an enzyme, capable of increasing or decreasing at least one metabolite, including any metabolite encompassed herein. In some embodiments, the one or more cellular therapies comprise a cell comprising a nucleic acid (RNA or DNA) encoding a protein and/or a protein capable of acting as a transcription factor. In some embodiments, the one or more cellular therapies comprise a cell comprising one or more non-mammalian proteins. The cell may be genetically modified to comprise the enzyme, protein, and/or nucleic acid. In some embodiments, the one or more cellular therapies comprise a cell genetically modified to overexpress an enzyme capable of increasing or decreasing at least one metabolite, including any metabolite encompassed herein. The enzyme may be endogenous or exogenous to the cell. Generation of the cell therapy may follow determination of an individual's ability to respond to a CAR-related therapy, or the cell therapy may reside in a repository, such as cryopreserved, and utilized upon determination of a need for the therapy.

Proteins, including enzymes and/or transcription factors, used in embodiments encompassed herein include, but are not limited to, ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (cIF5A), ATF4, and/or Gcn2.

Embodiments of the disclosure include methods for treating an individual comprising the steps of: (a) measuring a concentration of at least one metabolite in a blood sample (including a serum sample) from the individual to identify the individual as having a responder phenotype or as having a non-responder phenotype; and (b) administering either alone a therapeutically effective amount of a cellular therapy (including at least cells expressing one or more engineered receptors) to the individual, wherein the therapeutically effective amount is correlated with the individual having a responder or non-responder phenotype or in combination with other drugs or metabolites or chemical compounds as therapies to generate effective anti-tumor response or limiting toxicities related to cellular therapies. In specific embodiments, at least one metabolite comprises a metabolite selected from the group consisting of TMAO, indole, an indole derivative, 1-methylnicotinamide, nicotinamide, a polyamine, asymmetric dimethylarginine, symmetric dimethylarginine, taurine, and a combination thereof. Indole derivatives may comprise tryptophan, serotonin, indole-3-acetaldehyde, indole-3-lactate, indole-3-acetate, indoleacrylic acid, indoxyl sulfate, or a combination thereof. Polyamines may comprise diacetylspermine, N8-acetylspermidine, putrescine, spermidine, spermine, acetylputresceine, acetylspermidine, acetylspermine, N3AP, cadaverine, acetylcadaverine, diacetylcadaverine, or a combination thereof. In specific embodiments, a responder phenotype comprises a concentration of at least one metabolite in the blood sample that is statistically equal to a concentration of the metabolite in blood samples of individuals known to respond to an amount of the cellular therapy. In specific embodiments, a non-responder phenotype comprises a concentration of at least one metabolite in the blood sample that is statistically higher than a concentration of the metabolite in blood samples of individuals known to respond to an amount of the cellular therapy. A non-responder phenotype may comprise a concentration of at least one metabolite in the blood sample that is statistically lower than a concentration of the metabolite in blood samples of individuals known to respond to an amount of the cellular therapy. In particular cases, when the individual is identified as having a non-responder phenotype, the therapy is not provided to the individual. In some cases, when the individual is identified as having a non-responder phenotype, the cellular therapy is modulated. In some cases, when the individual is identified as having a non-responder phenotype, the cellular therapy is supplemented, such as with one or more metabolites or drugs. In specific embodiments of the method, at least one of the one or more metabolites was determined to be statistically lower than a concentration of the metabolite in blood samples of individuals known to respond to an amount of the cellular therapy. The cells of the cell therapy may be immune cells, such as T cell, NK cell, NKT cell, macrophage, or other hematopoietic cells. The cells of the cell therapy may express one or more engineered receptors, such as a chimeric antigen receptor, a non-native TCR, or both. The cell may further comprise a protein capable of increasing or decreasing at least one of the metabolites in the individual. The cell may be genetically modified to express the protein or metabolites. In specific embodiments, the protein comprises ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (cIF5A), ATF4, Gcn2, or a combination thereof. The method may further comprise administering a composition capable of increasing or decreasing at least one of the metabolites. When the composition is capable of increasing at least one of the metabolites, the composition may comprise at least one of the metabolites. In particular embodiments, the blood sample is from the individual prior to administering the cellular therapy. Methods include those further comprising measuring a concentration of the metabolite(s) in a second blood sample, the second blood sample comprising a blood sample from the individual after the individual has been administered the cellular therapy, wherein the individual has a responder phenotype when the concentration of the metabolite(s) in the blood sample are equal to the concentration of the metabolite(s) in the second blood sample. The method may further comprise measuring a concentration of the metabolite(s) in a second blood sample, the second blood sample comprising a blood sample from the individual after the individual has been administered the cellular therapy, wherein the individual has a non-responder phenotype when the concentration of the metabolite(s) in the blood sample are different from the concentration of the metabolite(s) in the second blood sample.

Embodiments of the disclosure include methods for decreasing the likelihood of or risk for toxicity (cytokine release syndrome, immune effector cell-associated neurotoxicity syndrome, prolonged cytopenias, hemophagocytic lymphohistiocytosis, or a combination thereof, for example) of a cellular therapy (including cells comprising one or more engineered receptors, such as a chimeric antigen receptor, a TCR, or both) comprising the steps of: (a) measuring a concentration of at least one metabolite in a blood sample (including a serum sample and that may be taken prior to the therapy) from the individual to identify the individual as having a responder phenotype or as having a non-responder phenotype; and (b) administering a therapeutically effective amount of the cellular therapy to the individual, wherein the therapeutically effective amount is dependent on the individual having a responder or non-responder phenotype. The individual may or may not be monitored for the toxicity. At least one metabolite may comprise a metabolite selected from the group consisting of an indole derivative, TMAO, tryptophan, serotonin, 1-methylnicotinamide, nicotinamide, a polyamine, asymmetric dimethylarginine, symmetric dimethylarginine, and a combination thereof. Indole derivatives include indole-3-acetaldehyde, indole, indoleacrylic acid, indole-3-lactate, indole-3-acetate, indoxyl sulfate, or a combination thereof. Polyamines may comprise diacetylspermine, N8-acetylspermidine, putrescine, spermidine, spermine, acetylputresceine, acetylspermidine, acetylspermine, N3AP, cadaverine, acetylcadaverine, diacetylcadaverine, or a combination thereof. A responder phenotype may comprise a concentration of at least one metabolite in the blood sample that is statistically equal to a concentration of the metabolite in blood samples of individuals known to respond to an amount of the cellular therapy. A non-responder phenotype may comprise a concentration of at least one metabolite in the blood sample that is statistically higher than a concentration of the metabolite in blood samples of individuals known to respond to an amount of the cellular therapy. In some cases, a non-responder phenotype comprises a concentration of at least one metabolite in the blood sample that is statistically lower than a concentration of the metabolite in blood samples of individuals known to respond to an amount of the cellular therapy. When the individual is identified as having a non-responder phenotype, the therapy may not be given. Cells of the therapy may be of any type, including a T cell, NK cell, NKT cell, macrophage, or other hematopoietic cells.

In some embodiments, the cells of the cell therapy further comprise a protein capable of increasing or decreasing at least one of the metabolites in the individual. The cells may be genetically modified to express one or more proteins, including at least ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (cIF5A), ATF4, Gcn2, or a combination thereof. In some cases, the method further comprises administering a composition capable of increasing or decreasing at least one of the metabolites. When the composition is capable of increasing at least one of the metabolites, the composition may comprise at least one of the metabolites.

In some embodiments, the method further comprises measuring a concentration of the metabolite(s) in a second blood sample, the second blood sample comprising a blood sample from the individual after the individual has been administered the cellular therapy, wherein the individual has a responder phenotype when the concentration of the metabolite(s) in the blood sample are equal to the concentration of the metabolite(s) in the second blood sample. The method may further comprise measuring a concentration of the metabolite(s) in a second blood sample, the second blood sample comprising a blood sample from the individual after the individual has been administered the cellular therapy, wherein the individual has a non-responder phenotype when the concentration of the metabolite(s) in the blood sample are different from the concentration of the metabolite(s) in the second blood sample.

In some embodiments, there are therapeutic compositions, comprising a cell expressing one or more engineered receptors (one or more chimeric antigen receptors and/or one or more non-native TCRs) and one or more metabolites as encompassed herein. The cells of the therapeutic compositions may comprise a genetic modification to express at least one protein capable of increasing or decreasing one or more metabolites. One or more metabolites may be selected from the group consisting of TMAO, indole, an indole derivative (e.g., tryptophan, serotonin, indole-3-acetaldehyde, indoleacrylic acid, indoxylsulfate, or a combination thereof), 1-methylnicotinamide, nicotinamide, a polyamine (e.g., diacetylspermine, N8-acetylspermidine, putrescine, spermidine, spermine, acetylputresceine, acetylspermidine, acetylspermine, N3AP, cadaverine, acetylcadaverine, diacetylcadaverine, or a combination thereof), asymmetric dimethylarginine, symmetric dimethylarginine, taurine, and a combination thereof. The protein may comprise ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (eIF5A), ATF4, Gcn2, or a combination thereof.

Embodiments of the disclosure include methods for treating an individual being administered a first cellular therapy comprising the steps of: (a) measuring a concentration of one or more metabolites in at least one blood sample from the individual; and (b) administering a therapeutic composition comprising metabolites, bacteria, a second cellular therapy, or a combination thereof when the concentration of the metabolite(s) is higher or lower than a baseline level. At least one metabolite may comprise a metabolite selected from the group consisting of TMAO, indole, an indole derivative (e.g., tryptophan, serotonin, indole-3-acetaldehyde, indoleacrylic acid, indoxyl sulfate, or a combination thereof), 1-methylnicotinamide, nicotinamide, a polyamine (e.g., diacetylspermine, N8-acetylspermidine, putrescine, spermidine, spermine, acetylputresceine, acetylspermidine, acetylspermine, N3AP, cadaverine, acetylcadaverine, diacetylcadaverine, or a combination thereof), asymmetric dimethylarginine, symmetric dimethylarginine, taurine, and a combination thereof. In some cases, a baseline level comprises a concentration of at least one metabolite in the blood sample of an individual known to respond to an amount of the cellular therapy. The first cellular therapy and/or the second cellular therapy may comprise cells expressing one or more engineered receptors (one or more chimeric antigen receptors, non-native TCRs, or both). Cells of the therapy may be of any type, including immune cells, such as T cells, NK cells, NKT cells, macrophages, or other hematopoietic cells. The second cellular therapy may further comprise a protein capable of increasing or decreasing at least one of the metabolites in the individual. Any cell therapy may be genetically modified to express one or more proteins, including ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (cIF5A), ATF4, Gcn2, or a combination thereof. The blood sample may comprise a serum sample. At least one blood sample may be from the individual prior to administering the first cellular therapy. The level of the metabolite may be at least 2-fold, 5-fold, 10-fold, 25-fold, 50-fold, 75-fold, 100-fold, 150-fold, 200-fold, 500-fold, 1000-fold, or 10000-fold higher or lower than a baseline level.

Embodiments of the disclosure include methods of predicting a response to a cellular therapy, comprising the steps of: (a) measuring a concentration of at least one metabolite in a blood sample from the individual; and (b) predicting a response to the cellular therapy when the metabolite is higher or lower than a baseline level. In specific embodiments, when the response is predicted to be deleterious to the individual, the cellular therapy is not administered to the individual, is modified prior to administering to the individual to make it less deleterious, the individual is given a different therapy, or a combination thereof. In specific embodiments, when the response is predicted to be deleterious to the individual, the individual is provided a therapeutically effective amount of (1) one or more metabolites, (2) a bacteria composition that can alter the metabolites or level thereof or that can alter other synthetic derivatives/products thereof or the level thereof, or (3) genetically modified immune cells that can alter the metabolites in the body to effect responses or toxicities to the therapy. In specific cases, when the response is not predicted to be deleterious to the individual, a therapeutically effective amount of the cellular therapy is administered to the individual. The level of the metabolite may be at least 2-fold, 5-fold, 10-fold, 25-fold, 50-fold, 75-fold, 100-fold, 150-fold, 200-fold, 500-fold, 1000-fold, or 10000-fold higher or lower than a baseline level.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIG. 1 shows serum metabolites associated with cytokine release syndrome (CRS);

FIG. 2 shows serum metabolites associated with immune effector cell-associated neurotoxicity syndrome (ICANS);

FIG. 3 shows association between circulating lysophospholipids and polyamines with progression free survival and overall survival in patients with B-cell lymphoma treated with CAR-T.

FIG. 4 provides development and validation of one example of a prognostic 6-marker metabolite panel for progression free survival and overall survival in patients with B-cell lymphoma treated with CAR-T.

FIG. 5A-5D show that B-cell lymphomas exhibit elevated mRNA expression of polyamine metabolizing enzymes and high spermidine synthase gene expression is prognostic for poor overall survival. 5A) Violin plots illustrating mRNA expression of polyamine-metabolizing enzymes (PMEs) in Diffuse Large B-cell lymphoma and normal B lymphocytes in the Basso lymphoma dataset. (Basso et al., 2005) Statistical significance was determined by 2-sided Wilcoxon rank sum test. Abbreviations: ODC1—ornithine decarboxylase 1; AMD1—adenosylmethionine decarboxylase 1; SRM—spermidine synthase; SMS—spermine synthase; SAT1—spermidine/spermine N1-acetyltransferase 1. 5B) Dot plots illustrating hazard ratios (HR) (95% CI) per unit increase in mRNA expression of PMEs and progression free survival (PFS) in The Cancer Genome Atlas (TCGA)-diffuse large B-cell lymphoma (DLBCL) transcriptomic dataset and overall survival in the Lenz (Lenz et al., 2008) and Shipp (Shipp et al., 2002) B-cell lymphoma transcriptomic datasets. 5C) Kaplan-Meier survival curves for association between mRNA expression of SRM> or ≤ an optimal change point value (Cecile Contal, 1999) and PFS in the TCGA-DLBCL dataset and overall survival in the Lenz (Lenz et al., 2008) and Shipp (Shipp et al., 2002) B-cell lymphoma datasets, respectively. 5D) Dot plots illustrate spearman rho coefficients (95% CI) for association between mRNA expression of polyamine metabolizing enzymes ODC1, SRM and SMS with gene-based signatures of immune-cell infiltrates and immune-checkpoint blockade related genes in the TCGA-DLBCL and Ma DLBCL (Ma et al., 2019) transcriptomic datasets. Gene based signatures were according to Bindea et al. (Bindea et al., 2013) Abbreviations: ODC1: ornithine decarboxylase 1; AMD1: adenosylmethionine decarboxylase 1; SRM: spermidine synthase; SMS: spermine synthase; SAT1: spermidine/spermine N1-acetyltransferase 1.

FIG. 6 shows progression free survival and overall survival curves for Responders and Non-responders in the Discovery and Validation Cohort.

FIG. 7 provides predictive performance of the 6-marker metabolite panel for discriminating CAR-T responders from non-responders.

FIG. 8 shows circulating levels of acetylated polyamines in patients with LBCL following CAR-T cell treatment.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. In specific embodiments, any embodiments herein may comprise, consist of, or consist essentially of any combination of markers. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” or “engineering” as used herein refers to an entity that is generated by the hand of man (or the process of generating same), including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they have reduced expression of one or more endogenous genes and/or because they express one or more heterologous genes (such as synthetic antigen receptors), in which case(s) the engineering is performed by the hand of man. With respect to an antigen receptor, the antigen receptor may be considered engineered because it comprises multiple components that are genetically recombined to be configured in a manner that is not found in nature, such as in the form of a fusion protein of components not found in nature so configured.

As used herein, the term “concentration” may be interchangeably used with the term “levels” when referring to the amount of a biomarker or metabolite present in an individual, including the amount of a biomarker or metabolite present in a biological sample from the individual. For example, the “level” of a measured metabolite may be used interchangeably, in some embodiments, with the “concentration” of the measured metabolite.

As used herein, when two values are “statistically” equal or different, the values are equal or different as determined by a statistical method suitable for comparing the values. For example, the level of a certain metabolite may be measured one or more times in a first biological sample to get a first value set for the metabolite in the first biological sample. The level of that metabolite may be measured on or more times in a second biological sample to get a second value set. To determine whether the first value set is statistically equal to, or different from, the second value set a statistical test, for example a Student's t-test, may be performed on the two sets. One skilled in the art can determine the resulting value, such as the resulting p-value, needed to reach statistical significance to determine whether the two value sets are statistically equal or different. Other statistical tests may be employed for more comparing multiple metabolites or comparing more than two value sets. In some embodiments, a p-value less than 0.1, 0.05, 0.01, 0.001, 0.0001, or less will determine that there is statistical significance between the one or more values sets.

As used herein, the term “therapeutically effective amount” is synonymous with “effective amount”, “therapeutically effective dose”, and/or “effective dose” and refers to the amount of a therapy that will elicit the biological or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays or interpretation of clinical data as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “individual” refers to an individual in need of a therapy. The individual can be a mammal, such as a human, dog, cat, horse, pig or rodent. The individual can be a patient, e.g., have or be suspected of having or at risk for having a disease or medical condition, including any cancer, such as hematological cancer or solid tumors. The hematological cancer may be any lymphoma, including B-cell lymphoma. The individual may have a disease or be suspected of having the disease, including any cancer. The individual may be asymptomatic. The individual may be of any gender. The individual may be of a certain age, such as at least 5, 10, 15, 20, 25, 30, 35, 40, 45, 50, 55, 60, 65, 70, 75, 80, 85, 90, 95, or 100 or more.

### II. General Embodiments

Embodiments disclosed herein show that plasma metabolomics correlate with efficacy and toxicities associated treating a disease, such as a cancer, with cellular therapies, including at least CAR-T therapies. The present disclosure relates to methods and compositions for the treatment of diseases, including cancer of any kind, by modulating certain metabolites to enhance the efficacy of cellular therapies, including CAR-T therapies. The present disclosure also relates to determination of plasma and serum metabolite abundance as a biomarker for the prediction of efficacy and/or toxicities related to CAR-T therapy. The cancer being treated by the adoptive cell therapy may be of any type and may be primary, relapsed, refractory, metastatic, and so forth. The cancer may be solid tumors or hematological malignancies, including lymphoma or leukemia. The predictive metabolite signatures

Embodiments of the disclosure include methods of identifying or predicting individuals that will or will not achieve an effective response to adoptive cellular therapy of any kind, including CAR-expressing cells (including T cells, NK cells, etc.). In specific embodiments, the methods predict individuals that are unlikely to achieve durable responses with adoptive cellular therapy of any kind. In various embodiments, the methods identify individuals that have a reduced chance of achieving effective response to adoptive cellular therapy, such as compared to individuals that are not so identified (e.g., lack the same marker(s) as the individual determined to have a reduced chance). In specific embodiments, the methods allow prediction of whether or not an individual will have a poor response to CAR-T cell therapy upon measuring one or more metabolites, including measuring one or more plasma metabolites. In specific cases, the metabolites include one or more polyamines and/or lysophospholipids, and in particular aspects the presence of elevated levels of several polyamines and/or lysophospholipids (e.g., compared to the general population) indicates that the individual would have worse progression free survival (PFS) and overall survival (OS), such as compared to an individual that did not have elevated levels of the polyamines and/or lysophospholipids.

In certain embodiments, one or more checkpoint inhibitor therapies are also utilized in any methods herein and can include one or more lymphoid checkpoint inhibitors including but not limited to PD-1, PDI-1, TIM3, LAG3 inhibitors and/or can include one or more myeloid checkpoint inhibitors including but not limited to CD47, SIRalpha inhibitors. Commercially approved drugs include nivolumab, pembrozulimab, ipilimumab, atezolizumab, etc., that may be utilized.

In specific cases, a response is effective when one or more symptoms are alleviated and/or when the onset of one or more symptoms is delayed. The efficacy may comprise reduction of the number of cancer cells (including to a non-detectable level), a reduction in tumor load, a reduction in tumor size, an improvement in the quality of life of the individual, an extension of life of the individual, a reduction in the risk of metastasis, prevention of metastasis, delaying the onset of metastasis, a combination thereof, and so forth. In some embodiments, an individual is also subjected to steps of determining the presence of marker(s) other than those encompassed herein, such as elevated lactate dehydrogenase (LDH), c-reactive protein (CRP), increased tumor interferon signaling, and/or elevated IL-6 at baseline, as examples.

In various embodiments, an individual that has the presence or absence of one or more certain markers in a sample from the individual is provided an effective amount of one or more treatments (e.g., surgery, chemotherapy, radiation, hormone therapy, drug therapy, antibodies, and so forth), and the individual is provided the treatment as a result of determination of the respective presence or absence of the one or more markers. The method may or may not include a step of determining that the individual has cancer.

Metabolites/proteins measurements and interventions to modulate cellular therapies, (including CAR-T therapies and the toxicities associated therein) to enhance efficacy of the therapy is useful. Embodiments herein encompass metabolomics methods, including metabolomics in blood, serum, or plasma samples from an individual. Such methods may be used in clinical decision-making in selecting or managing recipients of cellular therapies, including CAR-T therapies. Embodiments herein also encompass compositions, such as cellular therapies and/or metabolites. The cellular therapy may comprise CAR-T cells, in specific cases. The cellular therapy may comprise genetically engineered cells capable of expressing one or more enzymes for modulating metabolite levels. In some embodiments, individuals receiving a cellular therapy, such as a CAR-T therapy, may benefit from the compositions encompassed herein.

Included herein are methods of measuring or correlating one or more metabolites with efficacy or toxicity of a cellular therapy for which an individual is in need, wherein the levels of the one or more metabolites are determined from a sample from the individual. In specific embodiments, the methods include therapy steps in which a therapy is determined based on the measuring or correlating and in which the therapy is then administered. In specific embodiments, the level of one or more metabolites is assessed in an individual to facilitate clinical therapy decision making for the individual. In some cases, the therapy is delivered following the assessment, whereas in other cases the therapy is modified following the assessment and prior to administration to the individual. For example, following a determination that an individual may or will have reduced efficacy based on the presence of one or more certain metabolites, the cells of a CAR cell therapy may be changed to another type of cell and/or the cell may be genetically or otherwise modified.

In specific cases, the cell may be modified to express one or more particular proteins (including enzymes) and/or nucleic acids. The proteins and/or nucleic acids may be capable of altering, inside and/or outside of the cell, one or more metabolites, including any metabolite encompassed herein. The protein may comprise ornithine decarboxylase (ODC), arginase, lysine decarboxylase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (cIF5A), ATF4, Gcn2, or a combination thereof. In some embodiments, the cell comprises a nucleic acid encoding ornithine decarboxylase (ODC), arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (cIF5A), ATF4, Gcn2, or a combination thereof.

In addition or alternatively, the CAR may be modified, such as utilizing a different extracellular domain, transmembrane domain, and/or intracellular domain. In specific cases, the CAR may be modified to utilize a different scFv, a different transmembrane domain, and/or one or more different costimulatory domains. In addition, or alternatively, the therapy comprises the CAR-related cells but one or more other compositions are given in addition to the cells and including, for example, one or more metabolites that were determined to be deficient in level or that is suspected of being deficient in level. Methods of the disclosure include methods of predicting response to a cellular therapy for an individual. The individual may be in need of predicting whether or not the cellular therapy will be efficacious or toxic, for example, because the individual has cancer and is in need of CAR-related therapies wherein the CAR is directed against one or more antigens of the cancer of the individual. If based on analysis of one or more metabolites, including at least their level, it is determined that the CAR cellular therapy would not be efficacious and/or would toxic to the individual, then in some cases the individual is not given the CAR cellular therapy. In other cases, when it is determined that the CAR cellular therapy would not be efficacious and/or would be toxic to the individual, then the individual may be given a different cancer therapy. In some cases, when it is determined that the CAR cellular therapy would not be efficacious and/or would toxic to the individual, then the CAR cellular therapy is modified to be more efficacious and/or less toxic.

In certain embodiments, an individual is administered one or more metabolites, including any metabolite encompassed herein. Specific embodiments encompass methods including therapy steps in which a metabolite therapy is determined based on the measuring or correlating of one or more metabolites, and in which the metabolite therapy is then administered. In some cases, the metabolite therapy is delivered following the assessment, whereas in other cases the metabolite therapy is modified following the assessment and prior to administration to the individual. The metabolite therapy may comprise administering metabolites, for example orally, (including through diet), via injection (including at least intravenously), or any other suitable method for administering one or more metabolites. In specific embodiments, one or more metabolites encompassed herein are administered to enhance CAR T-cell therapy efficacy and/or reduce CAR T therapy toxicity.

In certain embodiments, an individual is administered one or more bacteria strains. Specific embodiments encompass methods including therapy steps in which a bacteria therapy is determined based on the measuring or correlating of one or more metabolites, and in which the bacteria therapy is then administered. In some cases, the bacteria therapy is delivered following the assessment, whereas in other cases the bacteria therapy is modified following the assessment and prior to administration to the individual. The bacteria therapy may comprise administering bacteria, for example orally, (including through diet), via injection (including intravenously), or any other suitable method for administering bacteria. The bacteria therapy may comprise any bacteria strain capable of increasing or decreasing a metabolite in an individual.

There are many ways of administering or manipulating these metabolites in the human body including but not limited to (1) administering the metabolite(s) directly, such as through i.v. or orally; (2) diet can also manipulate these metabolites; (3) administering bacteria that have modified genes that can change these metabolites; and/or (4) fecal microbiota transplantation (FMT) can modify these metabolites.

In some embodiments, a survival outcome for an individual with cancer is determined based on measuring of the level of one or more metabolites in an individual in need of therapy. The survival outcome may be determined based on the metabolite(s) levels in the absence of the therapy or as a result of the therapy. Once it is determined, the clinical course for therapy may or may not be altered depending on the outcome. The survival outcome may be determined to be positive such that the therapy is then given, or the survival outcome may be determined not to be positive such that the therapy is not given or is altered.

In some embodiments, a cellular therapy is monitored for efficacy by measuring levels of one or more metabolites in an individual receiving the therapy. In specific cases, the level of one or more metabolites may be determined prior to and following administration of the cellular therapy. When it is determined that one or more metabolites indicate that the cellular therapy is either losing efficacy or has lost efficacy or is at risk for becoming toxic or is toxic, then one or more measures can be taken to address the change. The monitoring of the metabolites may be at certain durations of time with the therapy, for example. In some cases, the monitoring includes measuring levels of one or more metabolites to assess the risk for or presence of CRS and/or ICANS.

### III. Measuring Metabolites

Certain embodiments of the disclosure encompass methods for detecting one or more metabolites in a biological sample from an individual, including measuring the level of one or more metabolites in a biological sample from an individual. Any method known in the art may be used to measure metabolites including, but not limited to, mass spectrometry, nuclear magnetic resonance (NMR), ion-mobility spectrometry, electrochemical detection, Raman spectroscopy, immunoassays, and/or radiolabeling. Any mass spectrometry method may be employed in embodiments herein including, but not limited to, time of flight, quadrupole, ion trap, Fourier-transform ion cyclotron resonance, electron ionization, atmospheric-pressure chemical ionization, electrospray ionization, matrix-assisted laser desorption/ionization, or a combination thereof. In some embodiments, metabolites in a sample are separated using any method including, but not limited to, chromatography (such as gas chromatography, liquid chromatography (such as high-performance liquid chromatography and ultra-high-performance liquid chromatography)), capillary electrophoresis, or a combination thereof. In some embodiments, metabolite separation and measurements are done in tandem, for example by GC-MS, LC-MS, LC-MS/MS, and/or HPLC-MS.

In some embodiments, the metabolites measured in a first biological sample are compared to normal values for the measured metabolites. In some embodiments, the normal values are determined by measuring the metabolites in a second biological sample from an individual known to have a desired phenotype. As a non-limiting example, certain metabolites are measured in the second biological sample from an individual known to have the desired phenotype of responding to a cellular therapy. In some embodiments, such individual has a responder phenotype. In some embodiments, the metabolite levels measured in said individual are a baseline level. The metabolite levels measured in said individual are then compared to the metabolite levels measured in the first biological sample. If the metabolite levels in the first and second biological sample are equal, including statistically equal, the individual from which the first biological sample was taken may be said to have the desired phenotype. If the metabolite levels in the first and second biological sample are different, including statistically different, the individual from which the first biological sample was taken may be said to not have the desired phenotype. In some embodiments, an individual said to not have a desired phenotype is identified as having a non-responder phenotype. In some cases, a normal value for one or more particular metabolites is based on values from a general population, which may be determined by standard means or may be known in the art, for example.

In some embodiments, the metabolites measured in a first biological sample are compared to normal values for the measured metabolites. In some embodiments, the normal values are determined by measuring baseline values of the metabolites. The baseline values may be measured at any point prior to or during a treatment, including a cellular therapy such as a CAR-T therapy. For example, a biological sample may be taken from an individual prior to the individual beginning a cellular therapy to establish a baseline measurement for one or more metabolites. In some embodiments, after establishing baseline levels of certain metabolites, the individual is administered at least one therapy, including any therapy (or combination of therapies) encompassed herein. After administering the therapy, at least one additional biological sample is taken from the individual. Certain metabolites, such as the metabolites measured in the first baseline sample, may be measured in the additional biological sample(s). The levels of each metabolite in each sample may then be compared.

In some embodiments, an individual may be said to have a responder phenotype when one or more metabolites are unchanged (such as the metabolites levels are equal, including statistically equal, or not different, including not statistically different) between the baseline sample and one or more of the additional samples. In some embodiments, an individual may be said to have a non-responder phenotype when the levels one or more metabolites from the baseline sample are different, including statistically different, from the levels in at least one of the additional samples.

Metabolites useful for the embodiments encompassed herein include, but are not limited to, TMAO, indoxyl sulfate, acetylcadaverine, 1-methylnicotinamide, diacetylspermine, Ng, Ng-dimethyl-1-arginine, 5,6-dihydrouridine, nicotinamide, 4,7-dioxo-octanoic acid, N8-acetylspermidine, plasphosphatidylcholine(o-40:7) and/or plasphosphatidylcholine(p-40:6), n-acetyl-1-phenylalanine, 2-hydroxyphenylacetic acid; 4-hydroxyphenylacetate, benzyl alcohol, phosphatidylcholine(37:5), 1-valine, phosphatidylcholine(38:5), l-norvaline, oleamide, norleucine, phosphatidylcholine(36:4), lysophosphatidylcholine(20:4), phosphatidylcholine(38:4); phosphatidylglycerol(40:0), sphingomyelin(38:1), adenosine 5′-monophosphate, 2′-deoxyguanosine 5′-monophosphate, cholesterol ester(20:4), taurine, 3-methoxy-1-tyrosine, 2′-deoxyguanosine 5′-diphosphate; adenosine 3′.5′-diphosphate; adenosine 5′-diphosphate, deoxycorticosterone acetate phosphatidylcholine(33:2), glucosylceramide(36:0), 2-hydroxypyridine, beta-nicotinamide adenine dinucleotide phosphate, azelaic acid, plasphosphatidylcholine(p-36:0), plasphosphatidylethanolamine(o-34:3) and/or plasphosphatidylethanolamine(p-34:2), 10-hydroxydecanoate, plas_phosphatidylethanolamine(o-40:6) and/or plas_phosphatidylethanolamine(p-40:5), plasphosphatidylcholine(p-33:2), 1-(hydroxymethyl)-5,5-dimethyl-2,4-imidazolidinedione_exogenous, phosphatidylethanolamine(44:11), plas_phosphatidylethanolamine(o-36:5) and/or plas_phosphatidylethanolamine(p-36:4), citrulline, plasphosphatidylethanolamine(o-40:6) and/or plasphosphatidylethanolamine(p-40:5), homoserine, thyroxine, n-olcoyl ethanolamine, plasphosphatidylcholine(o-30:1) and/or plasphosphatidylcholine(p-30:0), plasphosphatidylethanolamine(o-38:5) and/or plasphosphatidylethanolamine(p-38:4), lysophosphatidylethanolamine(18:2), nepsilon.nepsilon.nepsilon-trimethyllysine, plasphosphatidylethanolamine(o-36:5) and/or plasphosphatidylethanolamine(p-36:4), 8-methoxykynurenate, lysophosphatidylinositol(18:1), 1-n.gamma.-monomethylarginine, plas_phosphatidylethanolamine(0-40:7) and/or plas_phosphatidylethanolamine(p-40:6), lysophosphatidylcholine(17:2), I-asparagine, prostaglandin c2, sphingomyelin(34:0), creatine phosphate, lysophosphatidylcholine(18:2), glucosylceramide(40:1), 12(s)-hete; 15(s)-hete;5(s)-hete, lactosylceramide(18:1/16:0), plasphosphatidylethanolamine(o-38:4) and/or plasphosphatidylethanolamine(p-38:3), lactosylceramide(32:1), triacylglycerol(58:4), sphinganine, d-ribose 5-phosphate;quinate, deoxycarnitine, aspartyl-threonine, phosphatidylcholine(28:0), metformin, phosphatidylcholine(40:8), acylcarnitine(c18:0), phylloquinone, deoxycarnitine; phosphocholine, indole-3-acetaldehyde, 6-phosphogluconic acid, sphingomyelin(32:0), glycyl-threonine, acylcarnitine(c4:0), hypoxanthine, 1-methylhistidine; 3-methylhistidine; n(pai)-methyl-1-histidine, dl-5-hydroxylysine, indole, 1-methionine, ascorbate, prostaglandin f2a, serotonin; serotonin, glycerophosphocholine, 1-aminocyclopropane-1-carboxylate, phosphatidylethanolamine(36:1); phosphatidylethanolamine(36:1), ceramide(42:0), 3-amino-5-hydroxybenzoic acid, d-tryptophan; l-tryptophan, n-acetyl-dl-serine; o-acetyl-1-serine, phosphatidylinositol(43:2), triacylglycerol(47:0), ceramide(42:0)iso, 11-2.6-diaminoheptanedioate, pyridoxal 5′-phosphate, quinoline, indoleacrylic acid, l-anserine, methylpyrazine, o-butanoyl-r-carnitine, 2′-deoxyguanosine 5′-monophosphate; adenosine 5′-monophosphate; n-acetylneuraminate, urate, ceramide(40:0), 3-(4-hydroxyphenyl)propionic acid, glycerophosphocholine; sn-glycero-3-phosphocholine, n-methyl-d-aspartic acid, citrate, plaslysophosphatidylethanolamine(p-22:0), plaslysophosphatidylcholine(p-16:0), l-tryptophan, lysophosphatidylcholine(22:5), lysophosphatidylcholine(p-18:0/0:0) and/or lysophosphatidylcholine(0-18:1), 5′-deoxyadenosine diacetylspermine, triacylglycerol(51:6), (3′-sulfo)galß-cer(d18:1/18:0(2oh)), phosphatidylcholine(35:2), phosphatidylcholine(33:3), phosphatidylcholine(34:4), triacylglycerol(51:5), 4-pyridoxate, phosphatidylcholine(32:2), thymidine-5′-diphospho-alpha-d-glucose, nicotinamide mononucleotide, phosphatidylcholine(33:2), cl(1′-[15:0/15:0],3′-[15:0/16:1(92)])[rac], plasphosphatidylethanolamine(o-38:4) and/or plasphosphatidylethanolamine(p-38:3); plasphosphatidylethanolamine(o-40:7) and/or plasphosphatidylethanolamine(p-40:6), free fatty acid (14:1) (myristelaidic acid); free fatty acid (14:1) (myristoleic acid), 3-cis-hydroxy-b,e-caroten-3′-one, phosphatidylcholine(33:1), phosphatidylcholine(31:0), triacylglycerol(51:4), triacylglycerol(56:4), plasphosphatidylethanolamine(o-34:3) and/or plasphosphatidylethanolamine(p-34:2), galactosylceramide(36:1) and/or glucosylceramide(36:1), cis-quinceoxepane, plasphosphatidylethanolamine(o-38:5) and/or plasphosphatidylethanolamine(p-38:4), putative_kdnalpha2-3galbeta1-4glcbeta-cer(d18:1/24:0), plasphosphatidylethanolamine(o-36:5) and/or plasphosphatidylethanolamine(p-36:4), 1,2-didecanoyl-sn-glycero-3-phosphocholine, lysophosphatidylinositol(18:1), triacylglycerol(16:0_16:1_18:2), triacylglycerol(51:3), triacylglycerol(55:7), (r.r)-tartaric acid; (s.s)-tartaric acid, deoxyuridine, galß1-3 galß1-3 galß1-3 galß1-3 galß1-3 galß1-4 galß1-4glcß-cer(42:2), plasphosphatidylethanolamine(o-38:4) and/or plasphosphatidylethanolamine(p-38:3), lactosylceramide(32:1), ceramide(42:1)iso, sphinganine, d-ribose 5-phosphate; quinate, triacylglycerol(49:3), deoxycarnitine, n-alpha-acetyl-1-asparagine, phosphatidylcholine(40:8), phylloquinone, acetylcholine; deoxycarnitine, ceramide(43:1), triacylglycerol(49:2), phosphatidylcholine(35:4), d-glucono-1.5-lactone, lysophosphatidylcholine(15:0), triacylglycerol(56:3), lysophosphatidylcholine(o-16:2(9e,10e)/0:0)[u], ceramide(42:0), sphingomyelin(42:1), ceramide(42:0)iso, ceramide(40:0), n-methyl-d-aspartic acid, l-carnitine, triacylglycerol(61:6), or a combination thereof.

In some embodiments, one or more metabolites selected from the group consisting of TMAO, indoxyl sulfate, acetylcadaverine, 1-methylnicotinamide, diacetylspermine, Ng, Ng-dimethyl-1-arginine, 5,6-dihydrouridine, nicotinamide, 4,7-dioxo-octanoic acid, N8-acetylspermidine, plasphosphatidylcholine(o-40:7) and/or plasphosphatidylcholine(p-40:6), n-acetyl-1-phenylalanine, 2-hydroxyphenylacetic acid; 4-hydroxyphenylacetate, benzyl alcohol, phosphatidylcholine(37:5), 1-valine, phosphatidylcholine(38:5), 1-norvaline, oleamide, norleucine, phosphatidylcholine(36:4), lysophosphatidylcholine(20:4), phosphatidylcholine(38:4); phosphatidylglycerol(40:0), sphingomyelin(38:1), adenosine 5′-monophosphate, 2′-deoxyguanosine 5′-monophosphate, cholesterol ester(20:4), taurine, 3-methoxy-1-tyrosine, 2′-deoxyguanosine 5′-diphosphate; adenosine 3′,5′-diphosphate; adenosine 5′-diphosphate, deoxycorticosterone acetate, and a combination thereof are measured in an individual, including in a biological sample of the individual, to determine whether the individual has a desired phenotype. The desired phenotype may be a response to a cellular therapy (including a CAR-T cell therapy), such as a complete response or a durable complete response.

In some embodiments, one or more metabolites selected from the group consisting of phosphatidylcholine(33:2), glucosylceramide(36:0), 2-hydroxypyridine, beta-nicotinamide adenine dinucleotide phosphate, azelaic acid, plasphosphatidylcholine(p-36:0), plasphosphatidylethanolamine(o-34:3) and/or plasphosphatidylethanolamine(p-34:2), 10-hydroxydecanoate, plas_phosphatidylethanolamine(o-40:6) and/or plas_phosphatidylethanolamine(p-40:5), plasphosphatidylcholine(p-33:2), 1-(hydroxymethyl)-5,5-dimethyl-2,4-imidazolidinedione_exogenous, phosphatidylethanolamine(44:11), plas_phosphatidylethanolamine(o-36:5) and/or plas_phosphatidylethanolamine(p-36:4), citrulline, plasphosphatidylethanolamine(o-40:6) and/or plasphosphatidylethanolamine(p-40:5), homoserine, thyroxine, n-oleoyl ethanolamine, plasphosphatidylcholine(o-30:1) and/or plasphosphatidylcholine(p-30:0), plasphosphatidylethanolamine(o-38:5) and/or plasphosphatidylethanolamine(p-38:4), lysophosphatidylethanolamine(18:2), nepsilon.nepsilon.nepsilon-trimethyllysine, plasphosphatidylethanolamine(o-36:5) and/or plasphosphatidylethanolamine(p-36:4), 8-methoxykynurenate, lysophosphatidylinositol(18:1), 1-n.gamma.-monomethylarginine, plas_phosphatidylethanolamine(o-40:7) and/or plas_phosphatidylethanolamine(p-40:6), lysophosphatidylcholine(17:2), I-asparagine, prostaglandin c2, sphingomyelin(34:0), creatine phosphate, lysophosphatidylcholine(18:2), glucosylceramide(40:1), 12(s)-hete; 15(s)-hete;5(s)-hete, lactosylceramide(18:1/16:0), plasphosphatidylethanolamine(o-38:4) and/or plasphosphatidylethanolamine(p-38:3), lactosylceramide(32:1), triacylglycerol(58:4), sphinganine, d-ribose 5-phosphate; quinate, deoxycarnitine, aspartyl-threonine, phosphatidylcholine(28:0), metformin, phosphatidylcholine(40:8), acylcarnitine(c18:0), phylloquinone, deoxycarnitine; phosphocholine, indole-3-acetaldehyde, 6-phosphogluconic acid, sphingomyelin(32:0), glycyl-threonine, acylcarnitine(c4:0), hypoxanthine, 1-methylhistidine; 3-methylhistidine; n(pai)-methyl-1-histidine, dl-5-hydroxylysine, indole, I-methionine, ascorbate, prostaglandin f2a, serotonin; serotonin, glycerophosphocholine, 1-aminocyclopropane-1-carboxylate, phosphatidylethanolamine(36:1); phosphatidylethanolamine(36:1), ceramide(42:0), 3-amino-5-hydroxybenzoic acid, d-tryptophan; l-tryptophan, n-acetyl-dl-serine; o-acetyl-1-serine, phosphatidylinositol(43:2), triacylglycerol(47:0), ceramide(42:0)iso, 11-2.6-diaminoheptanedioate, pyridoxal 5′-phosphate, quinoline, indoleacrylic acid, I-anserine, methylpyrazine, o-butanoyl-r-carnitine, 2′-deoxyguanosine 5′-monophosphate; adenosine 5′-monophosphate; n-acetylneuraminate, urate, ceramide(40:0), 3-(4-hydroxyphenyl)propionic acid, glycerophosphocholine; sn-glycero-3-phosphocholine, n-methyl-d-aspartic acid, citrate, plaslysophosphatidylethanolamine(p-22:0), plaslysophosphatidylcholine(p-16:0), l-tryptophan, lysophosphatidylcholine(22:5), lysophosphatidylcholine(p-18:0/0:0) and/or lysophosphatidylcholine(o-18:1), 5′-deoxyadenosine, and a combination thereof are measured in an individual, including in a biological sample of the individual, to determine whether the individual has a desired phenotype. The desired phenotype may be developing or not developing CRS after administration of a cellular therapy, including any cellular therapy encompassed herein.

In some embodiments, one or more metabolites selected from the group consisting of diacetylspermine, triacylglycerol(51:6), (3′-sulfo)galß-cer(d18:1/18:0(2oh)), phosphatidylcholine(35:2), phosphatidylcholine(33:3), phosphatidylcholine(34:4), triacylglycerol(51:5), 4-pyridoxate, phosphatidylcholine(32:2), thymidine-5′-diphospho-alpha-d-glucose, nicotinamide mononucleotide, phosphatidylcholine(33:2), cl(1′-[15:0/15:0],3′-[15:0/16:1(9z)])[rac], plasphosphatidylethanolamine(o-38:4) and/or plasphosphatidylethanolamine(p-38:3); plasphosphatidylethanolamine(o-40:7) and/or plasphosphatidylethanolamine(p-40:6), free fatty acid (14:1) (myristelaidic acid); free fatty acid (14:1) (myristoleic acid), 3-cis-hydroxy-b,e-caroten-3′-one, phosphatidylcholine(33:1), phosphatidylcholine(31:0), triacylglycerol(51:4), triacylglycerol(56:4), plasphosphatidylethanolamine(o-34:3) and/or plasphosphatidylethanolamine(p-34:2), galactosylceramide(36:1) and/or glucosylceramide(36:1), cis-quinceoxepane, plasphosphatidylethanolamine(o-38:5) and/or plasphosphatidylethanolamine(p-38:4), putative_kdnalpha2-3galbeta1-4glcbeta-cer(d18:1/24:0), plasphosphatidylethanolamine(o-36:5) and/or plasphosphatidylethanolamine(p-36:4), 1.2-didecanoyl-sn-glycero-3-phosphocholine, lysophosphatidylinositol(18:1), triacylglycerol(16:0_16:1_18:2), triacylglycerol(51:3), triacylglycerol(55:7), (r,r)-tartaric acid; (s,s)-tartaric acid, deoxyuridine, galß1-3 galß1-3 galß1-3 galß1-3 galß1-3 galß1-4 galß1-4glcß-cer(42:2), plasphosphatidylethanolamine(o-38:4) and/or plasphosphatidylethanolamine(p-38:3), lactosylceramide(32:1), ceramide(42:1)iso, sphinganine, d-ribose 5-phosphate; quinate, triacylglycerol(49:3), deoxycarnitine, n-alpha-acetyl-1-asparagine, phosphatidylcholine(40:8), phylloquinone, acetylcholine; deoxycarnitine, ceramide(43:1), triacylglycerol(49:2), phosphatidylcholine(35:4), d-glucono-1.5-lactone, lysophosphatidylcholine(15:0), triacylglycerol(56:3), lysophosphatidylcholine(o-16:2(9e,10e)/0:0)[u], ceramide(42:0), sphingomyelin(42:1), ceramide(42:0)iso, ceramide(40:0), n-methyl-d-aspartic acid, l-carnitine, triacylglycerol(61:6), and a combination thereof are measured in an individual, including in a biological sample of the individual, to determine whether the individual has a desired phenotype. The desired phenotype may be developing or not developing ICANS after administration of a cellular therapy, including any cellular therapy encompassed herein.

The biological sample may comprise any biological sample from an individual suitable for measuring metabolites. The biological sample may comprise a blood sample (including whole blood, serum, and/or plasma), a urine sample, a saliva sample, a biopsy, a tissue sample, a tumor sample, a cerebrospinal fluid sample, or a combination thereof.

### IV. Sample Preparation

In certain aspects, methods involve obtaining a sample from a subject. The method may comprise obtaining a blood sample. In some embodiments, the methods of obtaining provided herein include methods of biopsy such as fine needle aspiration, core needle biopsy, vacuum assisted biopsy, incisional biopsy, excisional biopsy, punch biopsy, shave biopsy or skin biopsy. In other embodiments, the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. In certain embodiments, the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. Cellular Therapies

Certain embodiments encompass one or more cellular therapies. In some embodiments, the cellular therapy comprises cells comprising at least one engineered receptor. The engineered receptor may comprise a chimeric antigen receptor (CAR), including directed toward any cancer antigen; in some cases, the CAR is an anti-CD19 CAR. The engineered receptor may comprise a protein, including an enzyme and/or transcription factor, capable of increasing or decreasing one or more metabolites inside and/or outside of the cell. The cellular therapy may be useful in treating one or more cancers, including hematological cancers such as leukemia or lymphoma (including at least B-cell lymphoma). In some embodiments, the cellular therapy targets a cancer cell. In some embodiments, the cellular therapy targets CD19, such as by comprising an anti-CD19 CAR. In some embodiments, the cellular therapy comprises T cells, including T cells comprising one or more CARs.

In some embodiments, the cellular therapy comprises cells comprising one or more enzymes capable of increasing or decreasing at least one metabolite, including any metabolites encompassed herein. The cells may be genetically engineered or modified to increase or decrease the expression of the enzyme(s). The enzyme may be capable of increasing or decreasing a metabolite in an individual, including any individual encompassed herein, such as an individual having a responder or non-responder phenotypes. In some embodiments, the enzyme is endogenous to the cells. In some embodiments, the enzyme is exogenous to the cells. In some embodiments, the cells comprising the enzyme also comprise an engineered receptor. In some embodiments, the cells comprising the enzyme do not comprise an engineered receptor. The cells may be engineered by the hand of man to express one or more CARs and one or more proteins that are able to decrease or increase the levels of one or more metabolites in a recipient individual.

Certain embodiments relate to cells comprising polypeptides or nucleic acids of the disclosure. In some embodiments, the cell is an immune cell. The immune cell may be any type of immune cell, including any cell expressing CD3, such as T-helper cells, invariant natural killer T (INKT) cells, cytotoxic T cells, T-regulatory cells (Treg) gamma-delta T cells, natural-killer (NK) T cells, neutrophils, or macrophages. In some embodiments, the cell comprises a T cell, such as a CD4+ T cell or a CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### VI. Chimeric Antigen Receptors (CARs)

Embodiments of the disclosure include cellular therapies in which the cells express one or more CARs. In some embodiments, the CAR comprises: a) one or more intracellular signaling domains, b) a transmembrane domain, and c) an extracellular domain comprising one or more antigen binding regions.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including in some cases a CAR that has been humanized to reduce immunogenicity (hCAR), wherein the CAR comprises an intracellular signaling domain, a transmembrane domain, and an extracellular domain comprising one or more signaling motifs. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the disclosure includes a full-length CAR cDNA or coding region and includes vector that encode the CAR. In some cases, the antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells. The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, a CAR is constructed with a specificity for a particular antigen (or marker or ligand), such as an antigen expressed in a particular cell type to be targeted by adoptive therapy, e.g., a cancer antigen, and/or an antigen intended to induce a dampening response, such as an antigen expressed on a normal or non-diseased cell type. Thus, the CAR typically includes in its extracellular portion one or more antigen binding molecules, such as one or more antigen-binding fragments, domains, or portions, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (that may be referred to as an extracellular domain comprising an antigen binding region) comprises a cancer-associated antigen or a pathogen-specific antigen binding domain. A cancer associated antigen may be of any kind so long as it is expressed on the cell surface of cancer cells. Exemplary embodiments of antigens include CD19, CD70, HLA-G. CD38, CD123, CLL1. EBNA, CD123, HER2, CA-125, TRAIL/DR4, CD20, carcinoembryonic antigen, alphafetoprotein, CD56, AKT. Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12. MART-1, melanoma-associated antigen, BAGE, DAM-6, -10, GAGE-1, -2, -8, GAGE-3, -4, -5, -6, -7B. NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, ICE, MUC1, MUC2. Phosphoinositide 3-kinases (PI3Ks), TRK receptors, PRAME, P15, RU1, RU2, SART-1, SART-3. Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP. Annexin II, CDC27/m. TPI/mbcr-abl, BCR-ABL, interferon regulatory factor 4 (IRF4), ETV6/AML. LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2. ML-IAP. EpCAM, ERG (TMPRSS2 ETS fusion genc), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

Any one or more costimulatory domains may be utilized in the CAR, at least from CD28, 4-1BB, OX40, CD27, and so forth.

### VII. Genetic Engineering

Certain aspects of the present disclosure relate to methods and compositions for gene editing (also “genetic engineering”), useful in the generation of one or more genetic modifications in a cell. As used herein, a “genetic modification,” describes a region of a genome of a cell that has been altered from its native (i.e., endogenous) sequence. Various methods and systems for gene editing are known in the art and include, for example, zinc finger nuclease (ZFN)-based gene editing, transcription activator-like effector nuclease (TALEN)-based gene editing, and CRISPR/Cas-based gene editing. In some embodiments, methods of the present disclosure comprise CRISPR/Cas-based gene editing, which comprises the use of components of a CRISPR system, for example a guide RNA (gRNA) and a Cas nuclease.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. A Cas nuclease and a gRNA can be introduced into the cell indirectly via introduction of one or more nucleic acids (e.g., vectors) encoding for the Cas nuclease and/or the gRNA. A Cas nuclease and a gRNA can be introduced into the cell directly by introduction of a Cas nuclease protein and a gRNA molecule. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA may be targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% sequence complementarity along the length of the tracr mate sequence when optimally aligned.

One or more vectors driving expression of one or more elements of a CRISPR system can be introduced into a cell such that expression of the elements of the CRISPR system direct formation of a CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding a Cas protein (also “Cas nuclease”). Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (Cpf1), Csy1, Csy2, Csy3, Cse1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The Cas nuclease can be Cas9 (e.g., from S. pyogenes or S. pneumonia). The Cas nuclease can be Cas12a. The Cas nuclease can direct cleavage of one or both strands at the location of a target sequence, such as within the target sequence and/or within the complement of the target sequence. The vector can encode a Cas nuclease that is mutated with respect to a corresponding wild-type enzyme such that the mutated Cas nuclease lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is or is more than 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The Cas nuclease may be part of a fusion protein comprising one or more heterologous protein domains. A Cas nuclease fusion protein may comprise any additional protein sequence, and optionally a linker sequence between any two domains. Examples of protein domains that may be fused to a Cas nuclease, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A Cas nuclease may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a Cas nuclease are described in US 20110059502, incorporated herein by reference.

### VIII. Administration of Therapeutic Compositions

Certain embodiments encompass the administration of one or more therapeutic compositions (which may be referred to as “pharmaceutical compositions”), including after measuring one or more metabolites. In some embodiments, therapeutic compositions are administered to an individual, including before or after the individual has had one or more samples analyzed for level of one or more metabolites. Different aspects may involve administering an effective amount of a composition to an individual. In some embodiments, at least one cellular therapy, such as any cellular therapy herein, may be administered to the individual to protect against or treat a condition (e.g., cancer). In some embodiments, one or more metabolites are administered to an individual to protect against or treat a condition or toxicity. Any of the cellular therapies may be administered to the individual in combination, including sequentially or concurrently. In some cases, a CAR therapy is provided subsequent to and/or prior to and/or during administration of one or more metabolites, including one or more metabolites that are determined to be deficient in level based on sample analysis of the individual Any therapeutic compositions can be administered in combination with one or more additional therapeutic agents (e.g., one or more chemotherapeutics, one or more immunotherapeutics, one or more biotherapeutics, a combination thereof etc.). Such compositions will generally be dissolved or dispersed in a pharmaceutically acceptable carrier or aqueous medium.

In some embodiments, cellular therapies are administered to an individual at a dose between 1 million cells/kg to 1 billion cells/kg. In some embodiments, cellular therapies are administered to an individual at a dose of approximately 1 million cells/kg, 2 million cells/kg, 3 million cells/kg million cells/kg, 4 million cells/kg million cells/kg, 5 million cells/kg million cells/kg, 6 million cells/kg million cells/kg, 7 million cells/kg million cells/kg, 8 million cells/kg million cells/kg, 9 million cells/kg million cells/kg, 10 million cells/kg million cells/kg, 11 million cells/kg million cells/kg, 12 million cells/kg million cells/kg, 13 million cells/kg million cells/kg, 14 million cells/kg, 15 million cells/kg, 16 million cells/kg, 17 million cells/kg, 18 million cells/kg, 19 million cells/kg, 20 million cells/kg, 21 million cells/kg, 22 million cells/kg, 23 million cells/kg, 24 million cells/kg, 25 million cells/kg, 26 million cells/kg, 27 million cells/kg, 28 million cells/kg, 29 million cells/kg, 30 million cells/kg, 31 million cells/kg, 32 million cells/kg, 33 million cells/kg, 34 million cells/kg, 35 million cells/kg, 36 million cells/kg, 37 million cells/kg, 38 million cells/kg, 39 million cells/kg, 40 million cells/kg, 41 million cells/kg, 42 million cells/kg, 43 million cells/kg, 44 million cells/kg, 45 million cells/kg, 46 million cells/kg, 47 million cells/kg, 48 million cells/kg, 49 million cells/kg, 50 million cells/kg, 51 million cells/kg, 52 million cells/kg, 53 million cells/kg, 54 million cells/kg, 55 million cells/kg, 56 million cells/kg, 57 million cells/kg, 58 million cells/kg, 59 million cells/kg, 60 million cells/kg, 61 million cells/kg, 62 million cells/kg, 63 million cells/kg, 64 million cells/kg, 65 million cells/kg, 66 million cells/kg, 67 million cells/kg, 68 million cells/kg, 69 million cells/kg, 70 million cells/kg, 71 million cells/kg, 72 million cells/kg, 73 million cells/kg, 74 million cells/kg, 75 million cells/kg, 76 million cells/kg, 77 million cells/kg, 78 million cells/kg, 79 million cells/kg, 80 million cells/kg, 81 million cells/kg, 82 million cells/kg, 83 million cells/kg, 84 million cells/kg, 85 million cells/kg, 86 million cells/kg, 87 million cells/kg, 88 million cells/kg, 89 million cells/kg, 90 million cells/kg, 91 million cells/kg, 92 million cells/kg, 93 million cells/kg, 94 million cells/kg, 95 million cells/kg, 96 million cells/kg, 97 million cells/kg, 98 million cells/kg, 99 million cells/kg, 100 million cells/kg, 200 million cells/kg, 300 million cells/kg, 400 million cells/kg, 500 million cells/kg, 600 million cells/kg, 700 million cells/kg, 800 million cells/kg, 900 million cells/kg, or 1 billion cells/kg.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### IX. Kits

Certain aspects of the present disclosure also concern kits containing compositions of the disclosure or compositions to implement methods disclosed herein. In some embodiments, kits can be used to evaluate one or more metabolites. In specific embodiments, the kit comprises one or more metabolites or one or more reagents to produce one or more metabolites. The kit may comprise any means to obtain or analyze blood. The kit may comprise a vector of any kind, including viral (retroviral, lentiviral, adenoviral, or adeno-associated viral) or non-viral (plasmid, transposon, etc.), such as that encodes part or all of a CAR and/or one or more enzymes that increase or decrease metabolite(s) level. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more labels, probes, synthetic molecules or inhibitors, reagents, eluents, standards, or any value or range and combination derivable therein.

Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, reagents, synthetic molecules or inhibitors, eluents, standards, of the disclosure for prognostic or diagnostic applications are included as part of the disclosure. Specifically contemplated are any such molecules corresponding to any metabolites identified herein.

Embodiments of the disclosure include kits for analysis of a pathological sample by assessing a metabolite profile for a sample comprising. The kit can further comprise reagents for labeling metabolites in the sample. The kit may also include labeling reagents. Labeling reagents can include an amine-reactive dye, for example.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Metabolomics Predicts Durable Complete Response and Toxicities

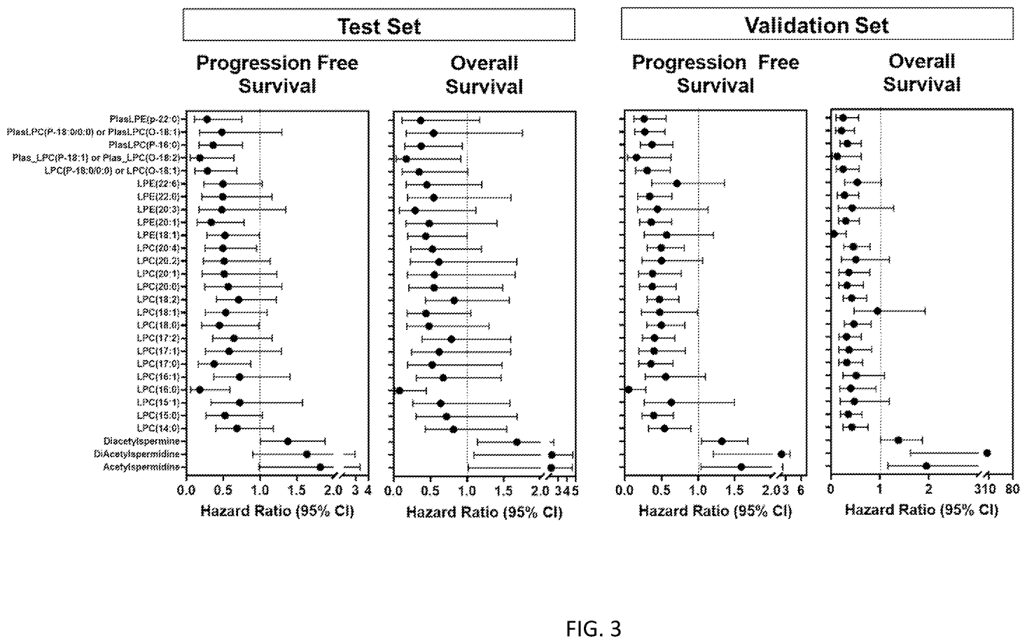

Baseline serum and plasma samples were collected from relapsed/refractory large B-cell lymphoma patients undergoing treatment with anti-CD19 CAR-T therapy. Metabolomic profiling was performed on baseline samples to determine association with response, survival outcomes, and toxicities. As shown in FIG. 3, certain plasma metabolites (including taurine, 1-methylnicotinamide, diacetylspermine, Ng-Ng-dimethyl-L-arginine, nicotinamide, and N8-acetylspermidine, TMAO, indoxyl sulfate, acetylcadaverine) were associated with poor or no complete response. Also, among the metabolites that were prognostic for either PFS or OS was high representation of polyamines and lysophospholipids. Elevated levels of the polyamines acetylspermidine (AcSpmd), diacetylspermidine (DiAcSpmd), and diacetylspermine (DAS) were found to be associated with worse PFS and OS whereas high levels of lysophospholipid species were conversely associated with favorable prognosis. The plasma samples from n=43 patients were collected at baseline prior to CAR-T infusion and untargeted metabolomics analyses performed using Xevo GS-X2 quadrupole time-of-flight (TOF) mass spectrometers (MS) under standardized operating procedures. FIG. 3 demonstrates a volcano plot illustrating area under the Receiver Operating Characteristic Curves (AUC) for individual annotated metabolites (x-axis) and −log(2-sided Wilcoxon Rank sum test p-value) (y-axis) for distinguishing patients with durable CR from those without at the 6 month follow-up. Increased polyamines were associated with poor responses in patients receiving CAR-T therapy. Especially, increased 1-methylnicotinamide and other correlated polyamines such as diacetylspermine and N8-acetylspermine are associated with poor responses. Moreover, taurine and other nucleotides were associated with increased responses. Taurine has been shown to correlate with proliferation of lymphocytes. High levels of lysophospholipids especially odd chain fatty acids were associated with good responses.

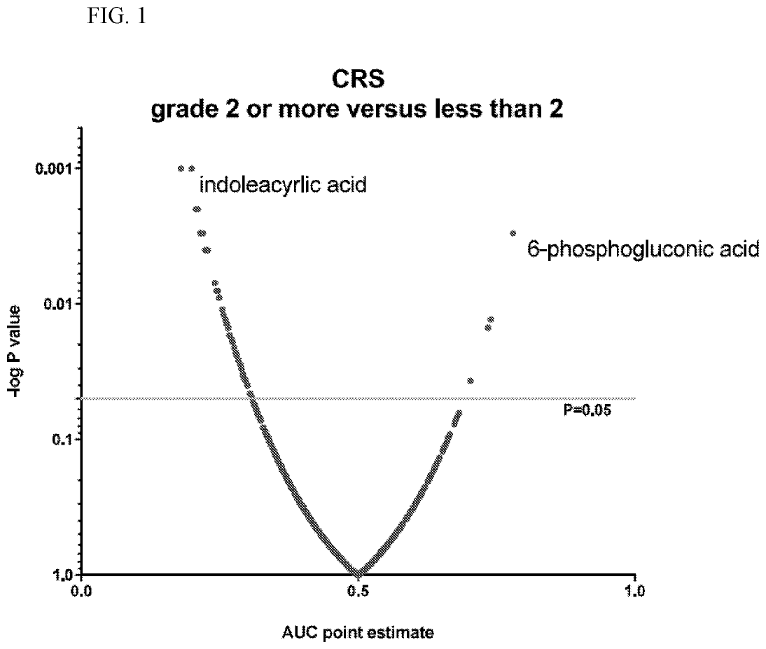

As shown in FIG. 1, certain metabolites (including indoleacrylic acid and 6-phosphogluconic acid) are predictive of cytokine release syndrome (CRS). The plasma samples from n=43 patients were collected at baseline prior to CAR-T infusion and untargeted metabolomics analyses performed using Xevo GS-X2 quadrupole time-of-flight (TOF) mass spectrometers (MS) under standardized operating procedures. FIG. 1 demonstrates a volcano plot illustrating area under the Receiver Operating Characteristic Curves (AUC) for individual annotated metabolites (x-axis) and −log(2-sided Wilcoxon Rank sum test p-value) (y-axis) for distinguishing patients with CRS from those without at the 6 month follow-up.

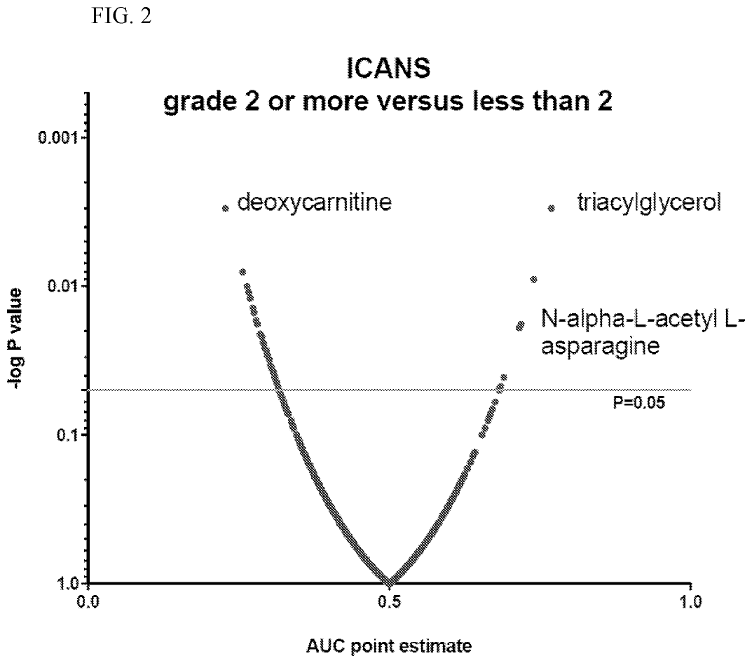

As shown in FIG. 2, certain metabolites (including deoxycarnitine, triacylglycerol, and N-alpha-L-acetyl L-asparagine) are predictive of immune effector cell-associated neurotoxicity syndrome (ICANS). The plasma samples from n=43 patients were collected at baseline prior to CAR-T infusion and untargeted metabolomics analyses performed using Xevo GS-X2 quadrupole time-of-flight (TOF) mass spectrometers (MS) under standardized operating procedures. FIG. 2 demonstrates a volcano plot illustrating area under the Receiver Operating Characteristic Curves (AUC) for individual annotated metabolites (x-axis) and −log(2-sided Wilcoxon Rank sum test p-value) (y-axis) for distinguishing patients with ICANS from those without at the 6 month follow-up.

### Example 2: Panel for Predicting Response to CAR-T Therapy

Focusing on polyamines and lysophospholipids, Cox proportional hazard models with LASSO regularization were used to select features and develop a 6-marker metabolite panel (M6P), consisting of AcSpmd, DiAcSpmd and 4 lysophospholipids (lysophosphatidylcholine(16:0), lysophosphatidylcholine(14:0), plasmanyllysophosphatidycholines (P-18:0 or O-18:1), and plasmanyl-lysophosphatidycholine(P-18:1 or O-18:2)), for predicting PFS. In multivariable Cox proportional hazard models, adjusting for other significant (2-sided p<0.05) variables, the M6P score yielded a hazard ratio (HR) of 3.65 (95% CI: 1.38-9.67) per unit increase. Metabolite abundances were assayed using mass spectrometry. In summary, plasma metabolomics are shown in embodiments herein to be a strong predictor of durability of responses and survival and toxicities after CAR T-cell therapy. The modulation of plasma metabolite levels has significant potential for influencing efficacy and toxicity after CAR T-cell therapy.

### Example 3: A Blood-Based Metabolite Signature Predictive of Aggressive Phenotype of B-Cell Lymphoma and Poor Response to CAR-T Therapy

Identification of Metabolite Biomarkers that Predict CAR-T Response and Model Development

A discovery cohort consisting of plasmas collected from 43 patients with r/r LBCL treated with anti-CD19 CAR-T therapy was assembled to profile for metabolite signatures predictive of response to CAR-T therapy. Patient and tumor characteristics are provided in Table 1. Axicabtagene ciloleucel and Tisagenlecleucel were the CAR-T product for 39 and 4 patients, respectively. There were no statistical differences in age, sex, stage, CRS or ICANs status, circulating LDH or CRP levels (Vercellino et al., 2020 between the two groups (Table 1). 3 patients died early due to toxicities before the response could be evaluated. Overall among the 40 evaluable patients, 15 (37%) of the 40 patients had an ongoing complete response (CR) and 24 (63%) had progressive disease(PD) at 6 months follow-up period, respectively. One remaining patient continued to be in ongoing partial response(PR) at 6 months follow-up period.

Multi-assay untargeted metabolomic analyses of these plasmas yielded 746 uniquely annotated metabolite features. Using Cox proportional hazard models, associations were screened for quantified metabolites with progression free survival and overall survival. Among the metabolites that were prognostic for either PFS or OS was high representation of polyamines and lysophospholipids. Elevated levels of the polyamines acetylspermidine (AcSpmd), diacetylspermidine (DiAcSpmd), and diacetylspermine (DAS) were found to be associated with worse progression free survival and overall survival, whereas high levels of lysophospholipid species were conversely associated with favorable prognosis.

Elevated levels of circulating polyamines have been shown to report on cancer status and associate with disease aggressiveness (Fahrmann, Bantis, et al., 2019; Fahrmann et al., 2021; Fahrmann Vykoukal et al., 2019). Lysophospholipids, particularly lysophosphatidylcholines, are bioactive lipids that are scavenged and metabolized by cancer cells to promote cancer cell growth (Fahrmann, Bantis et al., 2019; Kamphorst et al., 2013; Raynor et al., 2015). Decreased levels of lysophospholipids are frequently reported in plasmas of individuals presenting with various malignancies including pancreas, lung, ovarian and colorectal cancers (Fahrmann, Bantis et al., 2019; Kühn et al., 2016; Zhao et al., 2007).

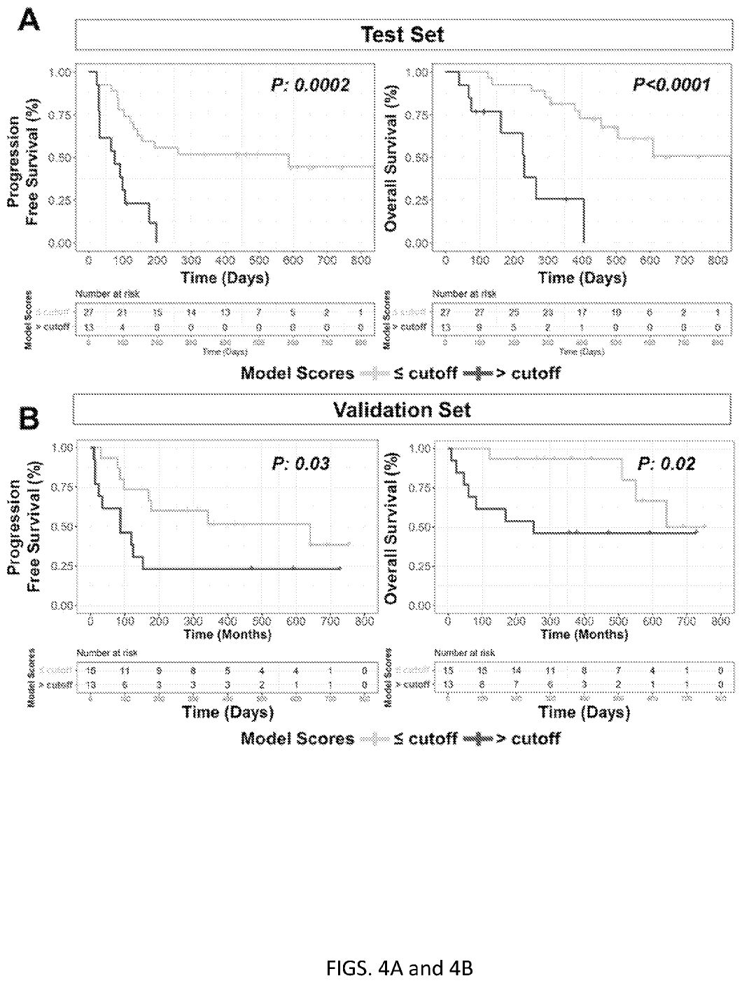

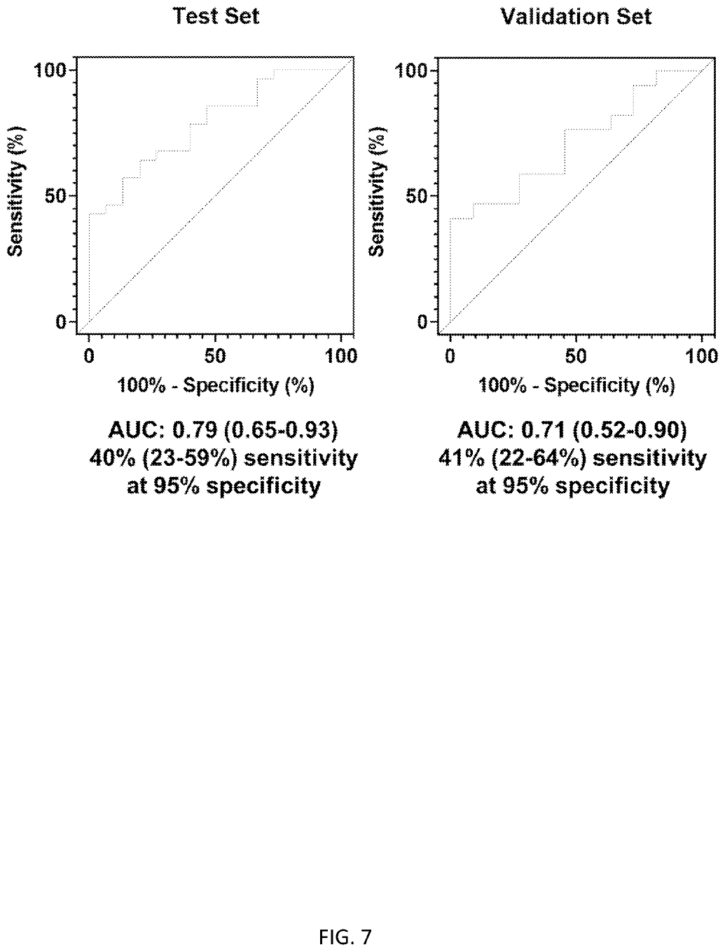

Focusing on polyamines and lysophospholipids, Cox proportional hazard models with LASSO regularization were used to select features and develop a biomarker panel for predicting progression free survival. The resultant 6-marker panel (6MetP), consisting of AcSpmd, DiAcSpmd and 4 lysophospholipids, yielded a hazard ratio (HR) of 4.53 (95% CI: 2.07-9.92) per unit increase for PFS in univariable cox proportional hazard models (FIG. 7). Nonproportionality hazard model tests yielded statistically nonsignificant P values. The 6MetP had an area under the Receiver Operating Characteristic curve of 0.79 (95% CI: 0.65-0.93) with 40% sensitivity at 95% specificity for discriminating individuals that were subsequently non-responsive to CAR-T from those individuals that were responsive to treatment (FIG. 7). Next, using log-rank test statistics from the Cox model, an optimal change point was calculated for the 6MetP to yield the greatest difference between individuals in the two already defined groups (disease progression versus no disease progression). Kaplan Meier survival curves demonstrated that patients with 6MP scores above the changepoint value had statistically significantly (Log rank Mantel Cox test 2-sided p<0.0001) worse PFS and worse OS (FIGS. 4A and 4B).

### Testing of the Metabolite Biomarker Panel in an Independent Test Set of Individuals Undergoing CAR-T Therapy

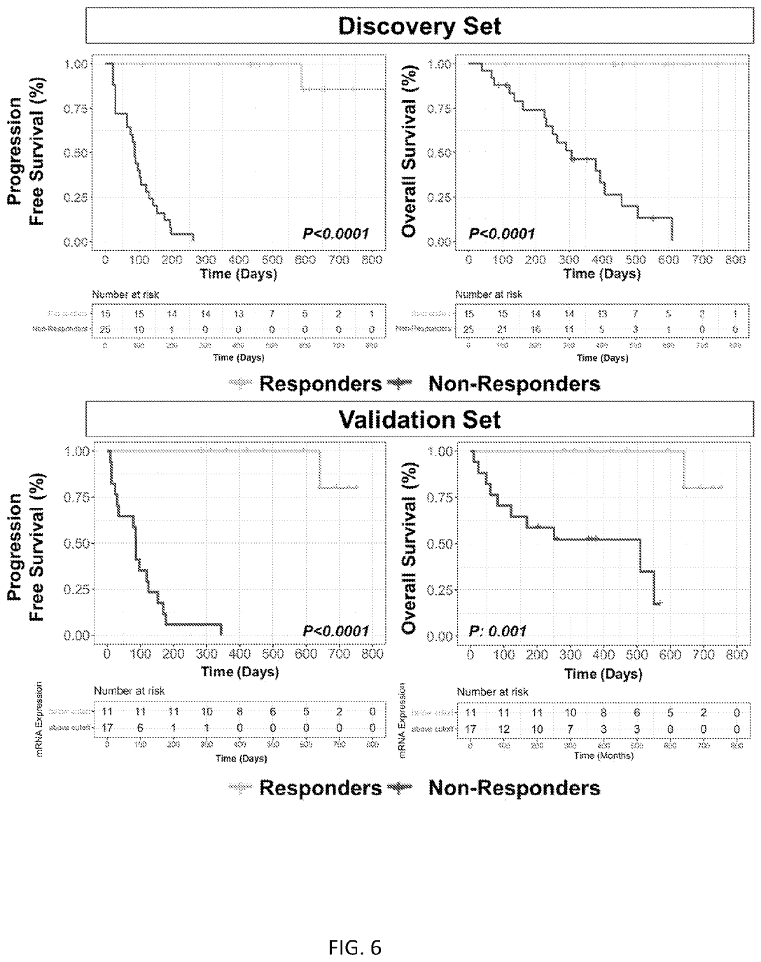

Testing of individual candidate metabolites as well as the 6MP panel using fixed coefficients and changepoint values developed in the discovery set for predicting PFS following anti-CD19 CAR-T treatment was performed in an independent test set consisting of 28 patients with r/rLBCL (Table 1). The median duration of follow-up in the validation cohort was 12 months (range 0.3-24.8 months). In the Validation Set, 11 (39%) of the 28 patients had an ongoing CR at 6 months of follow-up whereas the other 17 (61%) patients had PD or died. Median PFS and OS among non-responders was 2.9 and 8.25 months, respectively (FIG. 6). There were no statistically significant differences between the two groups with respect to age, sex, stage, CRS or ICANs status, circulating LDH or CRP levels. Patients that were non-responsive to CAR-T treatment did tend to have a higher (2+) ECOG status (Fisher's exact test 2-sided p: 0.04) (Table 1).

In the test set, elevated plasma polyamines were associated with poor PFS and OS, whereas elevated lysophospholipids were favorable prognostic indicators (FIG. 3). The fixed 6MetP yielded an AUC of 0.71 (95% CI: 0.52-0.90) with 41% sensitivity at 95% specificity for distinguishing patients that did not subsequently respond to treatment from those patients that had an objective response. A 6MetP score above the changepoint value established in the discovery set was found to be a statistically significant (Log rank Mantel Cox test 2-sided p<0.05) prognostic indicator of worse PFS and OS in the Test Set (FIGS. 4A and 4B).

Elevated mRNA Expression of Spermidine Synthase and Adenosylmethionine Decarboxylase 1 are Associated with Poor Overall Survival in Patients with B-Cell Lymphoma

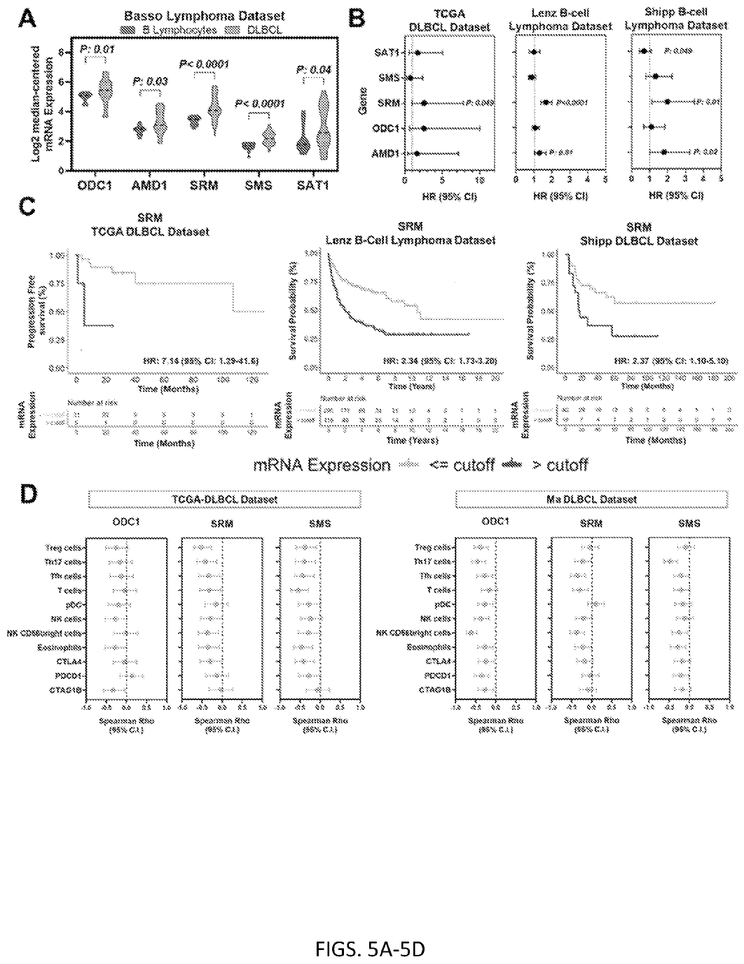

The analyses revealed that elevated levels of circulating polyamines are prognostic for poor PFS and OS among patients receiving CAR-T therapy. It was assessed whether circulating polyamine signature may reflect an aggressive phenotype that is less likely to be responsive to CAR-T treatment. To this end, the Basso Lymphoma gene expression dataset (Basso et al., 2005) was assessed and it was found that mRNA expression of polyamine metabolizing enzymes (PMEs) were statistically significantly higher (Wilcoxon rank sum test 2-sided p<0.05) in DLBCL cells compared to healthy B-lymphocytes (FIG. 5A), indicating that increased polyamine biosynthesis is a metabolic feature of DLBCL. Next, association was assessed between gene expression of PMEs and overall survival in two independent B-cell lymphoma transcriptomic datasets (Shipp et al., 2002; Lenz et al., 2008). In both datasets, elevated mRNA expression of spermidine synthase (SRM) and adenosylmethionine decarboxylase 1 (AMD1), a rate-limiting enzyme in polyamine metabolism, were statistically significantly associated with poor overall survival (FIG. 5B-5C). Thus, both plasma polyamines as well as mRNA expression of PMEs are concordantly associated with poor prognosis among individuals with B-cell lymphoma.

### Circulating Polyamine Levels Post CAR-T Cell Infusion in r/r LBCL Patients

Linear mixed models with random intercept and slope were incorporated to calculate the association between polyamine levels following CAR-T infusion. Reported values (slope and intercepts) in the table are the average representation of all calculated coefficients for each patient. P-values were calculated from 10,000 bootstraps of the delta value between responders and non-responders.

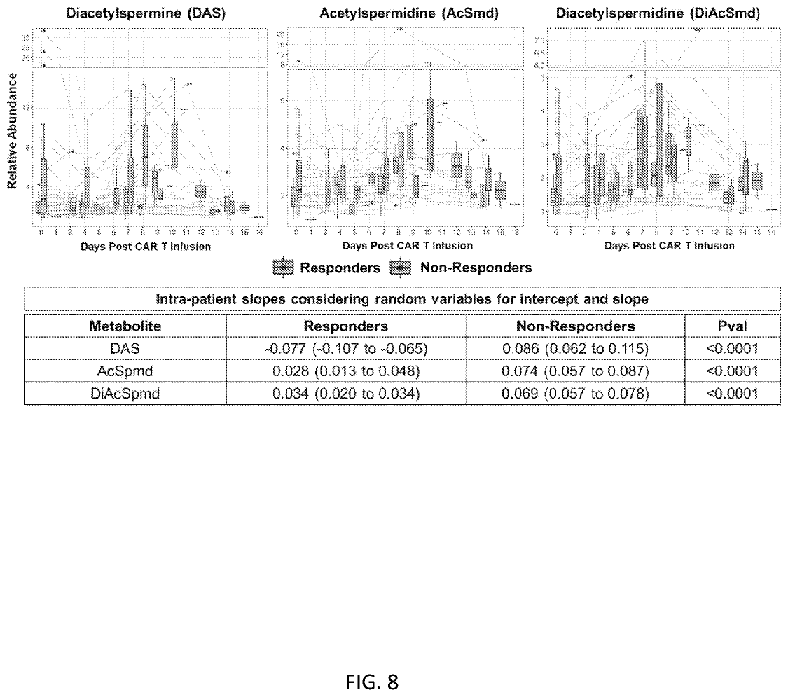

Intra-patient levels of AcSpmd, DiAcSpmd, and DAS were further assessed up to 16 days post CAR-T cell infusion, the results of which showed that polyamines remained high in patients who had progressive disease or died within 6 months post CAR T-cell treatment compared to those with an ongoing complete response. (FIG. 8)

TABLE 1
Patient and tumor characteristics for Discovery and Test Cohort.

| | Test Set | | | | Validation Set | | | |
|---|---|---|---|---|---|---|---|---|
| | On-going CR^{a} | | PD/PR^{a} | | On-going CR^{b} | | PD^{b} | |
| Participants, N | 15 | | 28 | | 11 | | 17 | |
| Age, mean +/− StDev | 57 +/− 16 | | 60 +/− 12 | | 65 +/− 14 | | 55 +/− 15 | |
| Sex, N (%) | | | | | | | | |
| Female | 2 | (13) | 11 | (39) | 7 | (64) | 5 | (29) |
| Male | 13 | (87) | 17 | (61) | 4 | (36) | 12 | (71) |
| Disease Type, N (%) | | | | | | | | |
| DLBCL | 13 | (87) | 21 | (75) | 8 | (73) | 13 | (76) |
| TFL | 2 | (13) | 7 | (25) | — | | — | |
| MCL | — | | — | | 2 | (18) | 2 | (12) |
| tCLL | — | | — | | — | | 1 | (6) |
| BCP-ALL | — | | — | | 1 | (9) | 1 | (6) |
| Stage, N (%) | | | | | | | | |
| I-II | 4 | (27) | 5 | (18) | 4 | (36) | 4 | (23) |
| III-IV | 11 | (73) | 23 | (82) | 6 | (55) | 12 | (71) |
| Unknown | — | | — | | 1 | (9) | 1 | (6) |
| Bulky Disease, N (%) | | | | | | | | |
| <10 cm | 8 | (53) | 20 | (71) | — | | — | |
| ≥10 cm | 3 | (20) | 3 | (11) | — | | — | |
| Unknown | 4 | (27) | 5 | (18) | — | | — | |
| ABC/GCB Status, N (%) | | | | | | | | |
| ABC | 3 | (20) | 7 | (25) | — | | — | |
| GCB | 7 | (47) | 16 | (57) | — | | — | |
| Unknown | 5 | (33) | 5 | (18) | — | | — | |
| Bcl-2/Bcl-6 expressors, N (%) | | | | | | | | |
| No | 5 | (33) | 9 | (32) | — | | — | |
| Yes | 5 | (33) | 11 | (39) | — | | — | |
| Unknown | 5 | (33) | 8 | (29) | — | | — | |
| Double/Triple Hit^{c}, N (%) | | | | | | | | |
| No | 9 | (60) | 15 | (54) | — | | — | |
| Yes | 1 | (7) | 7 | (25) | — | | — | |
| Unknown | 5 | (33) | 6 | (21) | — | | — | |
| ECOG at day 0, N (%) | | | | | | | | |
| 0 | 3 | (20) | 5 | (18) | 4 | (36) | 2 | (12) |
| 1 | 8 | (53) | 20 | (71) | 7 | (64) | 8 | (47) |
| 2 | 4 | (27) | 1 | (4) | — | | 4 | (23) |
| 3 | — | | 2 | (7) | — | | 3 | (18) |
| IPI Score | | | | | | | | |
| 0 | 1 | (7) | 1 | (4) | — | | — | |
| 1 | 3 | (20) | 4 | (14) | — | | — | |
| 2 | 6 | (40) | 7 | (25) | — | | — | |
| 3 | 3 | (20) | 10 | (36) | — | | — | |
| 4 | 2 | (13) | 6 | (21) | — | | — | |
| LDH (U/L) at day 0, | 276 +/− 110 | | 341 +/− 192 | | 231 +/− 86 | | 423+/− | |
| mean +/− StDev | | | | | | | | |
| CRS Grade, N (%) | | | | | | | | |
| 0 | 1 | (7) | 2 | (7) | — | | 2 | (12) |
| 1 | 8 | (53) | 14 | (50) | 7 | (64) | 7 | (41) |
| 2 | 6 | (40) | 10 | (36) | 3 | (27) | 5 | (29) |
| 3 | — | | 1 | (4) | 1 | (9) | 3 | (18) |
| 4 | — | | 1 | (4) | — | | — | |
| ICANs Grade, N (%) | | | | | | | | |
| 0 | 8 | (53) | 11 | (39) | 4 | (36) | 10 | (59) |
| 1 | 1 | (7) | 4 | (14) | 4 | (36) | 5 | (29) |
| 2 | 2 | (13) | 2 | (7) | 2 | (18) | 1 | (6) |

| | | | | | | | |
|---|---|---|---|---|---|---|---|
| 3 | 3 | (20) | 7 | (25) | — | 1 | (6) |

| | | | | | | | |
|---|---|---|---|---|---|---|---|
| 4 | 1 | (7) | 4 | (14) | 1 | (9) | — |

### Materials and Methods

### Human Subjects

The human plasma samples were collected through an international collaboration between MD Anderson Cancer Center (MDACC), Houston, USA and German Cancer Research Center (DKFZ), Heidelberg, Germany. The clinical data and patient's plasma samples were collected under existing Institutional research board (IRB) approved protocols at each center and conducted in accordance with institutional guidelines and the principles of the Declaration of Helsinki. Response status was determined by Lugano 2014 classification. CRS and ICANS were prospectively graded and managed according to the CAR-T-cell-therapy-associated toxicity guidelines. For MDACC cohort analysis, EDTA plasmas were obtained from 43 r/r LBCL patients on the day of CAR-T infusion (day 0). For validation purposes, another 20 patient plasma samples on day 0 of CAR-T therapy from German (DKFZ) r/r LBCL cohort were analyzed.

### Metabolomic Analysis

### Sample Extraction

### Primary Metabolites and Biogenic Amines

Plasma and serum metabolites were extracted from pre-aliquoted biospecimen (15 μL) with 45 μL of LCMS grade methanol (ThermoFisher) in a 96-well microplate (Eppendorf). Plates were heat sealed, vortexed for 5 min at 750 rpm, and centrifuged at 2000×g for 10 minutes at room temperature. The supernatant (30 μL) was carefully transferred to a 96-well plate, leaving behind the precipitated protein. The supernatant was further diluted with 60 μL of 100 mM ammonium formate, pH3 (Fisher Scientific). For Hydrophilic Interaction Liquid Chromatography (HILIC) positive ion analysis, 15 μL of the supernatant and ammonium formate mix were diluted with 195 μL of 1:3:8:144 water (GenPure ultrapure water system, Thermofisher): LCMS grade methanol (ThermoFisher): 100 mM ammonium formate, pH3 (Fisher Scientific): LCMS grade acetonitrile (ThermoFisher), whereas for HILIC negative ion analysis, 15 μL of the supernatant and ammonium formate mix were diluted with 90 μL of LCMS grade acetonitrile (ThermoFisher). For C18 analysis, 15 μL of the supernatant and ammonium formate mix were diluted with 90 μL water (GenPure ultrapure water system, Thermofisher) for positive and negative ion modes, respectively. Each sample solution was transferred to 384-well microplate (Eppendorf) for LCMS analysis.

### Complex Lipids

Pre-aliquoted serum or plasma samples (10 μL) were extracted with 30 μL of LCMS grade 2-propanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 90 μL of 1:3:2 100 mM ammonium formate, pH3 (Fischer Scientific): LCMS grade acetonitrile (ThermoFisher): LCMS grade 2-propanol (ThermoFisher) and transferred to a 384-well microplate (Eppendorf) for lipids analysis using LCMS.

### Untargeted Analysis of Primary Metabolites and Biogenic Amines

Untargeted metabolomics analysis was conducted on Waters Acquity™ UPLC system with 2D column regeneration configuration (I-class and H-class) coupled to a Xevo G2-XS quadrupole time-of-flight (qTOF) mass spectrometer. Chromatographic separation was performed using HILIC (Acquity™ UPLC BEH amide, 100 Å, 1.7 μm 2.1≤100 mm, Waters Corporation, Milford, U.S.A) and C18 (Acquity™ UPLC HSS T3, 100 Å, 1.8 μm, 2.1×100 mm, Water Corporation, Milford, U.S.A) columns at 45° C.

Quaternary solvent system mobile phases were (A) 0.1% formic acid in water, (B) 0.1% formic acid in acetonitrile and (D) 100 mM ammonium formate, pH 3. Samples were separated using the following gradient profile: for the HILIC separation a starting gradient of 95% B and 5% D was linearly changed to 70% A. 25% B and 5% D over a 5 min period at 0.4 mL/min flow rate, and to 100% A over 1 min, then followed by another 1 min isocratic gradient at 100% A at 0.4 mL/min flow rate to initiate the starting gradient for the next C18 run. For C18 separation, the chromatography gradient of was as follows: starting conditions, 100% A, with a linear change to 5% A, 95% B over a 5 min period at 0.4 mL/min flow rate, reverted back to 95% B, 5% D over 1 min, and then followed by 1 min isocratic gradient at 95% B, 5% D at 0.4 mL/min for the next HILIC run.

Binary pump was used for column regeneration and equilibration. The solvent system mobile phases were (A1) 100 mM ammonium formate, pH 3, (A2) 0.1% formic in 2-propanol and (B1) 0.1% formic acid in acetonitrile. The HILIC column was stripped using 90% A2 for 5 min at 0.25 mL/min flow rate, followed by 2 min equilibration using 100% B1 at 0.3 mL/min flow rate. Reverse phase C18 column regeneration was performed using 95% A1, 5% B1 for 2 min followed by column equilibration using 5% A1, 95% B1 for 5 min at 0.4 mL/min flow rate.

### Untargeted Analysis of Complex Lipids

For the lipidomic assay, untargeted metabolomics analysis was conducted on a Waters Acquity™ UPLC system coupled to a Xevo G2-XS quadrupole time-of-flight (qTOF) mass spectrometer. Chromatographic separation was performed using a C18 (Acquity™ UPLC HSS T3, 100 Å, 1.8 μm, 2.1×100 mm, Water Corporation, Milford, U.S.A) column at 55° C. The mobile phases were (A) water, (B) Acetonitrile, (C) 2-propanol and (D) 500 mM ammonium formate, pH 3. A starting elution gradient of 20% A, 30% B, 49% C and 1% D was linearly changed to 4% A, 14% B, 81% C and 1% D for 4.5 min, followed by isocratic elution at 4% A, 14% B, 81% C and 1% D for 2.1 min and column equilibration with initial conditions for 1.4 min.

### Mass Spectrometry Data Acquisition

Mass spectrometry data was acquired using ‘sensitivity’ mode in positive and negative electrospray ionization mode within 50-800 Da range for primary metabolites and 100-2000 Da for complex lipids. For the electrospray acquisition, the capillary voltage was set at 1.5 kV (positive), 3.0 kV (negative), sample cone voltage 30V, source temperature at 120° C., cone gas flow 50 L/h and desolvation gas flow rate of 800 L/h with scan time of 0.5 sec in continuum mode. Leucine Enkephalin; 556.2771 Da (positive) and 554.2615 Da (negative) was used for lockspray correction and scans were performed at 0.5 sec. The injection volume for each sample was 3 μL for complex lipids, and 6 μL for primary metabolites. The acquisition was carried out with instrument auto gain control to optimize instrument sensitivity over the samples acquisition time.

### Data Processing

LC-MS and LC-MSe data were processed using Progenesis QI (Nonlinear, Waters). Peak picking and retention time alignment of LC-MS and MSe data were performed using Progenesis QI software (Nonlinear, Waters). Data processing and peak annotations were performed using an in-house automated pipeline as previously described. Annotations were determined by matching accurate mass and retention times using customized libraries created from authentic standards and by matching experimental tandem mass spectrometry data against the NIST MSMS, LipidBlast or HMDB v3 theoretical fragmentations; for complex lipids retention time patterns characteristic of lipid subclasses was also considered. To correct for injection order drift, each feature was normalized using data from repeat injections of quality control samples collected every 10 injections throughout the run sequence. Measurement data were smoothed by Locally Weighted Scatterplot Smoothing (LOESS) signal correction (QC-RLSC) as previously described. Values are reported as ratios relative to the median of historical quality control reference samples run with every analytical batch for the given analyte.

### Statistical Analysis

Cox proportional hazard models with LASSO regularization using glmnet package in R statistical software were used to select metabolite features and develop a biomarker panel for predicting PFS. Coefficients of the selected features were derived in the Test Set and applied to the Validation Set. To test for the proportionality of Hazard assumption of a Cox regression, we utilized the method of Patricia et al.

Log rank statistic based methods as described by Contal and O'Quigley were used to determine optimal change point value for the model to distinguish patients that had progressive disease from those that had a complete response following CAR-T treatment. Kaplan-Meier survival analyses were performed using R Version 1.1.442. Log-rank (Mantel-Cox) tests were used to assess for statistical differences between survival curves.

Area under the Receiver Operating Characteristic curves (AUC) were generated using R (R version 3.6.0). The 95% confidence intervals presented for individual performance of each biomarker were based on the bootstrap procedure in which we re-sampled with replacement 1000 times. For two-class comparisons, statistical significance was determined using Wilcoxon rank sum test. Statistical significance was determined at p-values <0.05 for all analyses unless otherwise stated. Figures were generated in Graph Pad Prism Version 8.0 (GraphPad Software, Inc. San Diego, CA, USA).

### REFERENCES

The following references and those cited elsewhere herein, to the extent that they provide exemplary procedural or other details supplementary to those set forth herein, are specifically incorporated herein by reference.
Basso K, Margolin A A, Stolovitzky G, et al. Reverse engineering of regulatory networks in human B cells. Nat Genet 2005; 37:382-90.
Fahrmann J F, Bantis L E, Capello M, et al. A Plasma-Derived Protein-Metabolite Multiplexed Panel for Early-Stage Pancreatic Cancer. J Natl Cancer Inst 2019; 111:372-379.
Fahrmann J F, Irajizad E, Kobayashi M, et al. A MYC-Driven Plasma Polyamine Signature for Early Detection of Ovarian Cancer. Cancers (Basel) 2021; 13.
Fahrmann J F, Vykoukal J, Fleury A, et al. Association between plasma diacetylspermine and tumor spermine synthase with outcome in triple negative breast cancer. J Natl Cancer Inst 2019.
Kamphorst J J, Cross J R, Fan J, et al. Hypoxic and Ras-transformed cells support growth by scavenging unsaturated fatty acids from lysophospholipids. Proc Natl Acad Sci USA 2013; 110:8882-7.
Kühn T, Floegel A, Sookthai D, et al. Higher plasma levels of lysophosphatidylcholine 18:0 are related to a lower risk of common cancers in a prospective metabolomics study. BMC Med 2016; 14:13.
Lenz G, Wright G, Dave S S, et al. Stromal gene signatures in large-B-cell lymphomas. N Engl J Med 2008; 359:2313-23.
Raynor A, Jantscheff P, Ross T, et al. Saturated and mono-unsaturated lysophosphatidylcholine metabolism in tumour cells: a potential therapeutic target for preventing metastases. Lipids Health Dis 2015; 14:69.
Shipp M A, Ross K N, Tamayo P, et al. Diffuse large B-cell lymphoma outcome prediction by gene-expression profiling and supervised machine learning. Nat Med 2002; 8:68-74.
Vercellino L, Di Blasi R, Kanoun S, et al. Predictive factors of early progression after CAR T-cell therapy in relapsed/refractory diffuse large B-cell lymphoma. Blood Adv 2020; 4:5607-5615.
Zhao Z, Xiao Y, Elson P, et al. Plasma lysophosphatidylcholine levels: potential biomarkers for colorectal cancer. J Clin Oncol 2007; 25:2696-701.
Patricia M. Grambsch T M T: Proportional hazards tests and diagnostics based on weighted residuals. Biometrika 81:12, 1994
Cecile Contal J O Q: An application of changepoint methods in studying the effect of age on survival in breast cancer. Computational statistics & data analysis 30:253-270, 1999
Vykoukal J, Fahrmann J F, Gregg J R, et al: Caveolin-1-mediated sphingolipid oncometabolism underlies a metabolic vulnerability of prostate cancer. Nat Commun 11:4279, 2020
Friedman J, Hastie T, Tibshirani R: Regularization Paths for Generalized Linear Models via Coordinate Descent. J Stat Softw 33:1-22, 2010

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

It is contemplated that any method or composition described herein can be implemented with respect to any other method or composition described herein and that different embodiments may be combined. The claims originally filed are contemplated to cover claims that are multiply dependent on any filed claim or combination of filed claims.

What is claimed is:

1. A method for treating an individual comprising the steps of:
(a) measuring a concentration of at least one metabolite in a blood sample from the individual to identify the individual as having a responder phenotype or as having a non-responder phenotype; and
(b) administering a therapeutically effective amount of a cellular therapy to the individual, wherein the therapeutically effective amount is correlated with the individual having a responder or non-responder phenotype, said administering either alone or in combination with one or more metabolites, drugs, chemical compounds, biologics, and/or bacteria for modulating responses and/or reducing toxicities, wherein at least one metabolite comprises a metabolite selected from the group consisting of TMAO, indole, an indole derivative, 1-methylnicotinamide, nicotinamide, a polyamine, asymmetric dimethylarginine, symmetric dimethylarginine, taurine, a lysophospholipid, and a combination thereof.

2. The method of claim 1, wherein the indole derivative comprises tryptophan, serotonin, indole-3-acetaldehyde, indoleacrylic acid, indoxyl sulfate, indole-3-lactate, indole-3-acetate, or a combination thereof.

3. The method of claim 1 or 2, wherein the polyamine comprises diacetylspermine, N8-acetylspermidine, putrescine, spermidine, spermine, acetylputresceine, acetylspermidine, diacetylspermidine, acetylspermine, N3AP, cadaverine, acetylcadaverine, diacetylcadaverine, or a combination thereof.

4. The method of any one of claims 1-3, wherein the lysophospholipid comprises lysophosphatidyethanolamine, lysophosphatidylcholine, plasmanyl-lysophosphatidycholines, or a combination thereof.

5. The method of any one of claims 1-4, wherein a responder phenotype comprises a concentration of at least one metabolite in the blood sample that is statistically equal to a concentration of the metabolite in blood samples of individuals known to respond to an amount of the cellular therapy.

6. The method of any one of claims 1-4, wherein a non-responder phenotype comprises a concentration of at least one metabolite in the blood sample that is statistically higher than a concentration of the metabolite in blood samples of individuals known to respond to an amount of the cellular therapy.

7. The method of any one of claims 1-4, wherein a non-responder phenotype comprises a concentration of at least one metabolite in the blood sample that is statistically lower than a concentration of the metabolite in blood samples of individuals known to respond to an amount of the cellular therapy.

8. The method of any one of claims 1-7, wherein when the individual is identified as having a non-responder phenotype, the therapy is not provided to the individual.

9. The method of any one of claims 1-7, wherein when the individual is identified as having a non-responder phenotype, the cellular therapy is modulated.

10. The method of any one of claims 1-9, wherein when the individual is identified as having a non-responder phenotype, the cellular therapy is supplemented.

11. The method of claim 10, wherein the cellular therapy is supplemented with one or more metabolites.

12. The method of claim 11, wherein at least one of the one or more metabolites was determined to be statistically lower than a concentration of the metabolite in blood samples of individuals known to respond to an amount of the cellular therapy.

13. The method of any one of claims 1-12, wherein the cellular therapy comprises cells expressing one or more engineered receptors.

14. The method of claim 13, wherein the cell is an immune cell, T cell, NK cell, NKT cell, macrophage, or other hematopoietic cells.

15. The method of claim 13 or 14, wherein the engineered receptor comprises a chimeric antigen receptor, a TCR, or both.

16. The method of any one of claims 1-15, wherein the cellular therapy comprises CAR-T cell therapy.

17. The method of any one of claims 13-15, wherein the cell further comprises a protein capable of increasing or decreasing at least one of the metabolites in the individual.

18. The method of claim 17, wherein the cell is genetically modified to express the protein.

19. The method of claim 17 or 18, wherein the protein comprises ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (eIF5A), ATF4, Gcn2, or a mutated form of each said protein, or a combination thereof.

20. The method of any one of claims 1-19, further comprising administering a composition capable of increasing or decreasing at least one of the metabolites.

21. The method of claim 20, wherein when the composition is capable of increasing at least one of the metabolites, the composition comprises at least one of the metabolites.

22. The method of any one of claims 1-21, further comprising the step of providing to the individual an effective amount of an agent for modulating responses or reducing toxicities.

23. The method of claim 22, wherein the agent comprises a metabolite, drug, or chemical compound, or a combination thereof.

24. The method of claim 22, wherein the agent comprises a polyamine metabolism modulator or a lysophospholipid metabolism modulator, or a combination thereof.

25. The method of claim 24, wherein the polyamine metabolism modulator comprises an ODC1 inhibitor or a polyamine transporter inhibitor, a spermine synthase inhibitor, or a combination thereof.

26. The method of claim 25, wherein the ODC1 inhibitor is difluoromethylornithine (DFMO) and/or wherein the spermine synthase inhibitor is SBP-101 (diethyl dihydroxyhomospermine.

27. The method of claim 25, wherein the polyamine transporter inhibitor is AMXT-1501.

28. The method of claim 24, wherein the lysophospholipid metabolism modulator comprises an alkyl-lysophopholipid, such as edelfosine.

29. The method of claim 22-28, further comprising the administering of one or more checkpoint therapies with one or more of the metabolites.

30. The method of any one of claims 1-29, wherein the blood sample comprises a serum sample.

31. The method of any one of claims 1-30, wherein the blood sample is from the individual prior to administering the cellular therapy.

32. The method of claim 31, further comprising measuring a concentration of the metabolite(s) in a second blood sample, the second blood sample comprising a blood sample from the individual after the individual has been administered the cellular therapy, wherein the individual has a responder phenotype when the concentration of the metabolite(s) in the blood sample are equal to the concentration of the metabolite(s) in the second blood sample.

33. The method of claim 31, further comprising measuring a concentration of the metabolite(s) in a second blood sample, the second blood sample comprising a blood sample from the individual after the individual has been administered the cellular therapy, wherein the individual has a non-responder phenotype when the concentration of the metabolite(s) in the blood sample are different from the concentration of the metabolite(s) in the second blood sample.

34. A method for decreasing the likelihood of or risk for toxicity of a cellular therapy comprising the steps of:
(a) measuring a concentration of at least one metabolite in a blood sample from the individual to identify the individual as having a responder phenotype or as having a non-responder phenotype; and
(b) administering a therapeutically effective amount of the cellular therapy to the individual, wherein the therapeutically effective amount is dependent on the individual having a responder or non-responder phenotype, wherein at least one metabolite comprises a metabolite selected from the group consisting of an indole derivative, tryptophan, serotonin, 1-methylnicotinamide, nicotinamide, a polyamine, asymmetric dimethylarginine, symmetric dimethylarginine, taurine, a lysophospholipid, and a combination thereof.

35. The method of claim 34, wherein further comprising the step of determining a need for the cellular therapy.

36. The method of claim 4, wherein the indole derivative comprises indole-3-acetaldehyde, indole, indoleacrylic acid, indole-3-lactate, indole-3-acetate, or a combination thereof.

37. The method of claim 354, wherein the polyamine comprises diacetylspermine, N8-acetylspermidine, putrescine, spermidine, spermine, acetylputresceine, acetylspermidine, diacetylspermidine, acetylspermine, N3AP, cadaverine, acetylcadaverine, diacetylcadaverine, or a combination thereof.

38. The method of any one of claims 34-37, wherein a responder phenotype comprises a concentration of at least one metabolite in the blood sample that is statistically equal to a concentration of the metabolite in blood samples of individuals known to respond to an amount of the cellular therapy.

39. The method of any one of claims 34-37, wherein a non-responder phenotype comprises a concentration of at least one metabolite in the blood sample that is statistically higher than a concentration of the metabolite in blood samples of individuals known to respond to an amount of the cellular therapy.

40. The method of any one of claims 34-37, wherein a non-responder phenotype comprises a concentration of at least one metabolite in the blood sample that is statistically lower than a concentration of the metabolite in blood samples of individuals known to respond to an amount of the cellular therapy.

41. The method of any one of claims 34-40, wherein when the individual is identified as having a non-responder phenotype, the therapy is not administered.

42. The method of any one of claims 34-41, wherein the cellular therapy comprises a cell comprising one or more engineered receptors.

43. The method of claim 42, wherein the cell comprises an immune cell, T cell, NK cell, NKT cell, macrophage, or other hematopoietic cells.

44. The method of claim 42 or 43, wherein the engineered receptor comprises a chimeric antigen receptor, a TCR, or both.

45. The method of any one of claims 42-44, wherein the cell further comprises a protein capable of increasing or decreasing at least one of the metabolites in the individual.

46. The method of claim 45, wherein the cell is genetically modified to express the protein.

47. The method of claim 45 or 46, wherein the protein comprises ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (eIF5A), ATF4, Gcn2, or a mutated form of each said protein, or a combination thereof.

48. The method of any one of claims 34-47, further comprising administering a composition capable of increasing or decreasing at least one of the metabolites.

49. The method of claim 47, wherein when the composition is capable of increasing at least one of the metabolites, the composition comprises at least one of the metabolites.

50. The method of any one of claims 34-49, wherein the toxicity comprises cytokine release syndrome, immune effector cell-associated neurotoxicity syndrome, prolonged cytopenias, hemophagocytic lymphohistiocytosis, or a combination thereof.

51. The method of any one of claims 34-50, wherein the individual is monitored for the toxicity.

52. The method of any one of claims 34-51, wherein the blood sample comprises a serum sample.

53. The method of any one of claims 34-52, wherein the blood sample is from the individual prior to administering the cellular therapy.

54. The method of claim 53, further comprising measuring a concentration of the metabolite(s) in a second blood sample, the second blood sample comprising a blood sample from the individual after the individual has been administered the cellular therapy, wherein the individual has a responder phenotype when the concentration of the metabolite(s) in the blood sample are equal to the concentration of the metabolite(s) in the second blood sample.

55. The method of claim 53, further comprising measuring a concentration of the metabolite(s) in a second blood sample, the second blood sample comprising a blood sample from the individual after the individual has been administered the cellular therapy, wherein the individual has a non-responder phenotype when the concentration of the metabolite(s) in the blood sample are different from the concentration of the metabolite(s) in the second blood sample.

56. A therapeutic composition, comprising:
a cell expressing an engineered receptor and:
one or more metabolites.

57. The therapeutic composition of claim 56, wherein the cell comprises a genetic modification to express at least one protein capable of increasing or decreasing one or more metabolites.

58. The therapeutic composition of claim 56 or 57, wherein the one or more metabolites are selected from the group consisting of TMAO, indole, an indole derivative, 1-methylnicotinamide, nicotinamide, a polyamine, asymmetric dimethylarginine, symmetric dimethylarginine, taurine, a lysophospholipid, and a combination thereof.

59. The therapeutic composition of claim 58, wherein the indole derivative comprises tryptophan, serotonin, indole-3-acetaldehyde, indoleacrylic acid, indoxyl sulfate, indole-3-lactate, indole-3-acetate, or a combination thereof.

60. The therapeutic composition of claim 58, wherein the polyamine comprises diacetylspermine, N8-acetylspermidine, putrescine, spermidine, spermine, acetylputresceine, acetylspermidine, diacetylspermidine, acetylspermine, N3AP, cadaverine, acetylcadaverine, diacetylcadaverine, or a combination thereof.

61. The therapeutic composition of any one of claims 56-60, wherein the engineered receptor comprises one or more chimeric antigen receptors.

62. The therapeutic composition of any one of claims 57-61, wherein the protein comprises ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (eIF5A), ATF4, Gcn2, or a mutated form of each said protein, or a combination thereof.

63. A method for treating an individual being administered a first cellular therapy comprising the steps of:
(a) measuring a concentration of one or more metabolites in at least one blood sample from the individual; and
(b) administering a therapeutic composition comprising metabolites, bacteria, a second cellular therapy, or a combination thereof when the concentration of the metabolite(s) is higher or lower than a baseline level, wherein at least one metabolite comprises a metabolite selected from the group consisting of TMAO, indole, an indole derivative, 1-methylnicotinamide, nicotinamide, a polyamine, asymmetric dimethylarginine, symmetric dimethylarginine, taurine, a lysophospholipid, and a combination thereof.

64. The method of claim 63, further comprising the step of determining a need for the cellular therapy.

65. The method of claim 64, wherein the indole derivative comprises tryptophan, serotonin, indole-3-acetaldehyde, indoleacrylic acid, indoxyl sulfate, indole-3-lactate, indole-3-acetate, or a combination thereof.

66. The method of claim 64, wherein the polyamine comprises diacetylspermine, N8-acetylspermidine, putrescine, spermidine, spermine, acetylputresceine, acetylspermidine, diacetylspermidine, acetylspermine, N3AP, cadaverine, acetylcadaverine, diacetylcadaverine, or a combination thereof.

67. The method of any one of claims 63-66, wherein a baseline level comprises a concentration of at least one metabolite in the blood sample of an individual known to respond to an amount of the cellular therapy.

68. The method of any one of claims 63-67, wherein the first cellular therapy and/or the second cellular therapy comprises cells expressing one or more engineered receptors.

69. The method of claim 68, wherein the cell is an immune cell, T cell, NK cell, NKT cell, macrophage, or other hematopoietic cells.

70. The method of claim 68 or 69, wherein the engineered receptor comprises a chimeric antigen receptor, a TCR, or both.

71. The method of any one of claims 68-70, wherein the second cellular therapy further comprises a protein capable of increasing or decreasing at least one of the metabolites in the individual.

72. The method of claim 71, wherein the cell is genetically modified to express the protein.

73. The method of claim 71 or 72, wherein the protein comprises ornithine decarboxylase (ODC), lysine decarboxylase, arginase, agmatinase, spermidine synthase, spermine synthase, spermine oxidase, spermidine/spermine-N1-acetyltransferase (SSAT1), S-adenosyl-methionine decarboxylase (AdoMetDC), deoxyhypusine synthase (DHPS), deoxyhypusine hydroxylase (DOHH), antizyme inhibitors (AZI), eukaryotic initiation factor 5A (eIF5A), ATF4, Gcn2, or a mutated form of each said protein, or a combination thereof.

74. The method of any one of claims 63-74, wherein the blood sample comprises a serum sample.

75. The method of any one of claims 63-75, wherein at least one blood sample is from the individual prior to administering the first cellular therapy.

76. The method of any one of claims 63-75, wherein the level of the metabolite is at least 2-fold, 5-fold, 10-fold, 25-fold, 50-fold, 75-fold, 100-fold, 150-fold, 200-fold, 500-fold, 1000-fold, or 10000-fold higher or lower than a baseline level.

77. A method of predicting a response to a cellular therapy, comprising the steps of:
(a) measuring a concentration of at least one metabolite in a blood sample from the individual; and
(b) predicting a response to the cellular therapy when the metabolite is higher or lower than a baseline level, wherein at least one metabolite comprises a metabolite selected from the group consisting of TMAO, indole, an indole derivative, 1-methylnicotinamide, nicotinamide, a polyamine, asymmetric dimethylarginine, symmetric dimethylarginine, taurine, a lysophospholipid, and a combination thereof.

78. The method of claim 77, wherein when the response is predicted to be deleterious to the individual, the cellular therapy is not administered to the individual, is modified prior to administering to the individual to make it less deleterious, the individual is given a different therapy, or a combination thereof.

79. The method of claim 77 or 78, wherein when the response is predicted to be deleterious to the individual, the individual is provided a therapeutically effective amount of (1) one or more metabolites, (2) a bacteria composition that can alter the metabolites or level thereof or that can alter other synthetic derivatives/products thereof or the level thereof, or (3) genetically modified immune cells that can alter the metabolites in the body to effect responses or toxicities to the therapy.

80. The method of claim 77, wherein when the response is not predicted to be deleterious to the individual, a therapeutically effective amount of the cellular therapy is administered to the individual.

81. The method of any one of claims 77-80, wherein the level of the metabolite is at least 2-fold, 5-fold, 10-fold, 25-fold, 50-fold, 75-fold, 100-fold, 150-fold, 200-fold, 500-fold, 1000-fold, or 10000-fold higher or lower than a baseline level.
